SEQUENCE LISTING (8 sequences)

SEQ ID NO: 1 - DNA, 10 bp, Artificial Sequence
  Description of Artificial Sequence Synthetic primer
  modified_base (1)..(3): a, c, g, t, unknown or other
  modified_base (8)..(10): a, c, g, t, unknown or other
nnngccgnnn

SEQ ID NO: 2 - DNA, 10 bp, Artificial Sequence
  Description of Artificial Sequence Synthetic primer
  modified_base (1)..(3): a, c, g, t, unknown or other
  modified_base (8)..(10): a, c, g, t, unknown or other
nnngaagnnn

SEQ ID NO: 3 - DNA, 10 bp, Artificial Sequence
  Description of Artificial Sequence Synthetic primer
  modified_base (1)..(3): a, c, g, t, unknown or other
  modified_base (8)..(10): a, c, g, t, unknown or other
nnngagannn

SEQ ID NO: 4 - DNA, 10 bp, Artificial Sequence
  Description of Artificial Sequence Synthetic primer
  modified_base (1)..(3): a, c, g, t, unknown or other
  modified_base (8)..(10): a, c, g, t, unknown or other
nnntcctnnn

SEQ ID NO: 5 - DNA, 10 bp, Artificial Sequence
  Description of Artificial Sequence Synthetic primer
  modified_base (1)..(3): a, c, g, t, unknown or other
  modified_base (8)..(10): a, c, g, t, unknown or other
nnnttttnnn

SEQ ID NO: 6 - DNA, 10 bp, Artificial Sequence
  Description of Artificial Sequence Synthetic primer
  modified_base (1)..(3): a, c, g, t, unknown or other
  modified_base (8)..(10): a, c, g, t, unknown or other
nnnccccnnn

SEQ ID NO: 7 - DNA, 10 bp, Artificial Sequence
  Description of Artificial Sequence Synthetic primer
  modified_base (3)..(8): a, c, g, t, unknown or other
ctnnnnnntc

SEQ ID NO: 8 - DNA, 10 bp, Artificial Sequence
  Description of Artificial Sequence Synthetic primer
  modified_base (1)..(2): a, c, g, t, unknown or other
  modified_base (9)..(10): a, c, g, t, unknown or other
nnttagggnn

(19) United States
(12) Patent Application Publication
(10) Pub. No.: US 2007/0020667 A1
(43) Pub. Date: Jan. 25, 2007

(54) Methods and compositions for amplifying nucleic acids

(76) Inventors: David W. Ruff, San Francisco, CA (US); Mark E. Shannon, San Francisco, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/478,840

(22) Filed: Jun. 29, 2006

Related U.S. Application Data
(60) Provisional application No. 60/695,899, filed on Jun. 30, 2005.

(51) Int. Cl. C12Q 1/68; C07H 21/04; C12P 19/34

(52) U.S. Cl. 435/6; 435/91.2; 536/24.3

(57) ABSTRACT

Methods and compositions for amplifying nucleic acids are provided.

This application claims the benefit of U.S. Provisional Application No. 60/695,899, filed Jun. 30, 2005, which is incorporated by reference herein for any purpose.

### FIELD

Methods and compositions for amplifying nucleic acids are provided.

### INTRODUCTION

An amplification reaction is useful in certain research, diagnostic, medical, forensic and industrial fields. In certain instances, an amplification reaction generates amplification products for use in downstream assays. In certain instances, an amplification reaction generates reaction products for forensic or diagnostic purposes.

### SUMMARY

In certain embodiments, a method of amplifying a plurality of target nucleic acid sequences is provided. In certain embodiments, a reaction composition is formed comprising (a) a plurality of target nucleic acid sequences, (b) at least one set of primers, and (c) at least one DNA polymerase. In certain embodiments, the reaction composition is incubated under conditions wherein one or more of the plurality of target nucleic acid sequences are amplified. In certain embodiments, at least one of the at least one set of primers comprises a plurality of primers, wherein each primer of the plurality of primers comprises at least one designed portion and at least one random portion. In certain embodiments, one of the at least one designed portions consists of two nucleotides at the 5′ portion of the primer.

In certain embodiments, a composition for amplifying a plurality of target nucleic acid sequences is provided. In certain embodiments, the composition for amplifying a plurality of target nucleic acid sequences comprises (a) a plurality of target nucleic acid sequences; (b) at least one set of primers; and (c) at least one DNA polymerase. In certain embodiments, at least one of the at least one set of primers comprises a plurality of primers, wherein each primer of the plurality of primers comprises at least one designed portion and at least one random portion. In certain embodiments, one of the at least one designed portions consists of two nucleotides at the 5′ portion of the primer.

In certain embodiments, a method of amplifying a plurality of target nucleic acid sequences is provided. In certain embodiments, a reaction composition is formed comprising (a) a plurality of target nucleic acid sequences comprising a first position and a second position, (b) at least one set of primers, and (c) at least one DNA polymerase. In certain embodiments, the reaction composition is incubated under amplification conditions wherein one or more of the plurality of target nucleic acid sequences are amplified to form amplification products. In certain embodiments, at least one of the at least one set of primers comprises a plurality of primers, wherein each primer of the plurality of primers comprises at least one designed portion and at least one random portion. In certain embodiments, the at least one set of primers produces a first amplification product comprising the first position and a second amplification product comprising the second position. In certain embodiments, the amount of first amplification product produced with the at least one set of primers is greater than the amount of first amplification product produced with random primers incubated under the amplification conditions with the plurality of target nucleic acid sequences and the at least one polymerase. In certain embodiments, the amount of second amplification product produced with the at least one set of primers is greater than the amount of second amplification product produced with random primers incubated under the amplification conditions with the plurality of target nucleic acid sequences and the at least one polymerase.

In certain embodiments, a method of determining similarity between a plurality of target nucleic acid sequences from one or more sources and one or more reference sequences is provided. In certain embodiments, a reaction composition is formed comprising (a) a plurality of target nucleic acid sequences from one or more sources, (b) at least one set of primers, and (c) at least one DNA polymerase; In certain embodiments, the reaction composition is incubated under conditions wherein one or more of the plurality of target nucleic acid sequences are amplified to form amplification products. In certain embodiments, at least one of the at least one set of primers comprises a plurality of primers, wherein each primer of the plurality of primers comprises at least one designed portion and at least one random portion. In certain embodiments, one of the at least one designed portions consists of two nucleotides at the 5′ portion of the primer. In certain embodiments, sequence information of the amplification products is compared with sequence information of the one or more reference sequences to identify similarities and differences between the sequence information of the amplification products and the sequence information of the one or more reference sequences. In certain embodiments, each of the one or more reference sequences comprises a plurality of nucleic acid sequences. In certain embodiments, the similarity between a plurality of target nucleic acid sequences from one or more sources and the one or more reference sequences is determined based on the identified similarities and differences.

In certain embodiments, a method of amplifying a plurality of target nucleic acid sequences is provided. In certain embodiments, a reaction composition is formed comprising (a) a plurality of target nucleic acid sequences, (b) at least one set of primers, and (c) at least one DNA polymerase. In certain embodiments, the reaction composition is incubated under conditions wherein one or more of the plurality of target nucleic acid sequences are amplified. In certain embodiments, at least one of the at least one set of primers comprises a plurality of primers, wherein each primer of the plurality of primers comprises at least one designed portion and at least one random portion. In certain embodiments, one of the at least one designed portions comprises the 5′ portion of the primer.

In certain embodiments, a method of amplifying a plurality of target nucleic acid sequences is provided. In certain embodiments, a reaction composition is formed comprising (a) a plurality of target nucleic acid sequences, (b) at least one set of primers, and (c) at least one DNA polymerase. In certain embodiments, the reaction composition is incubated under conditions wherein one or more of the plurality of target nucleic acid sequences are amplified. In certain embodiments, at least one of the at least one set of primers comprises a plurality of primers, wherein each primer of the plurality of primers comprises at least one designed portion and at least one random portion. In certain embodiments, one of the at least one designed portions comprises the 3′ portion of the primer.

In certain embodiments, a method of amplifying a plurality of target nucleic acid sequences is provided. In certain embodiments, a reaction composition is formed comprising (a) a plurality of target nucleic acid sequences, (b) at least one set of primers, and (c) at least one DNA polymerase. In certain embodiments, the reaction composition is incubated under conditions wherein one or more of the plurality of target nucleic acid sequences are amplified. In certain embodiments, at least one of the at least one set of primers comprises a plurality of primers, wherein each primer of the plurality of primers comprises at least one designed portion and at least one random portion. In certain embodiments, at least one of the at least one designed portions is not a constant portion.

These and other features of the present teachings are set forth herein.

### DRAWINGS

The skilled artisan will understand that the drawings, described below, are for illustration purposes only. The figures are not intended to limit the scope of the invention in any way.

FIG. 1 depicts reaction products on an agarose gel as described in Example 2.

FIG. 2 depicts a picogreen standard curve as described in Example 3.

FIG. 3 depicts an Rnase P TaqMan Assay standard curve as described in Example 4.

FIG. 4 depicts reaction products on an agarose gel as described in Example 5.

FIG. 5 depicts amplification totals for “hotstart” amplification reactions versus “cold start” amplification reactions, as described in Example 6.

FIG. 6 depicts reaction products on an agarose gel as described in Example 7.

FIG. 7 depicts amplification results for primers DR01B to DR36B, as described in Example 8. FIG. 7 also depicts amplification results for DR03, DR04, DR07, and DR11, as described in Example 8.

FIG. 8 depicts amplification products on an agarose gel as described in Example 8. FIG. 8 also depicts amplification products, which were generated with one of primers DR01B to DR36B, on an agarose gel as described in Example 8.

FIG. 9 depicts fold amplification in a 22 hour time course experiment as described in Example 9. Fold amplification was calculated for a series of amplification reactions using different primer sets. The time course measured fold amplification at 0 hours, 2 hours, 5 hours, 8 hours, and 22 hours.

FIG. 10 depicts the relative hybridization positions on Chromosome 6 of 24 different TaqMan probes, as described in Example 10.

FIG. 11 depicts amplification results for seven different amplification reactions, where each different amplification reaction comprises a different primer set.

FIG. 11 also depicts fold amplification of amplification products for each of the seven different amplification reactions, as measured by 24 different TaqMan probes which hybridize at 24 different positions across human chromosome 6, as described in Example 10.

FIG. 12 depicts a graph showing fold amplification at 24 different positions for three primer sets, as described in Example 10.

FIG. 13 depicts amplification results for primers DR01C to DR26C, as described in Example 11.

FIG. 14 depicts amplification products on an agarose gel, as described in Example 11.

FIGS. 15A and 15B depict amplification results for amplification reactions with one of primer sets DR01D to DR14D. FIGS. 15A and 15B also depict fold amplification of amplification products for each of the different amplification reactions, as measured by 7 different TaqMan probes which hybridize at 7 different positions across the human genome.

FIG. 16 depicts fold amplification graphed versus GC content for primers DR01D to DR14D, as described in Example 12.

FIGS. 17A and 17B depict amplification results for primers DR1E to 80E, as described in Example 13.

FIGS. 18A and 18B depict amplification results for primers DR1F to DR62F, as described in Example 14.

FIG. 19 depicts amplification results of a cDNA library, as described in Example 15.

FIG. 20 graphically depicts the amplification results of Oligo dT₁₆ primed cDNAs, as described in Example 15.

FIG. 21 depicts fold amplification and fold difference results for 6 different primer sets under certain reaction conditions, as described in Example 19.

FIG. 22 graphically depicts fold amplification and fold difference for 6 different primer sets under certain reaction conditions, as described in Example 19.

### DESCRIPTION OF VARIOUS EMBODIMENTS

It is to be understood that both the foregoing general description and the following detailed description are exemplary and explanatory only and are not restrictive of the invention, as claimed. In this application, the use of the singular includes the plural unless specifically stated otherwise. In this application, the use of “or” means “and/or” unless specifically stated otherwise. Furthermore, the use of the term “including”, as well as other forms, such as “includes” and “included”, is not limiting.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. In the event that one or more of the incorporated literature and similar materials defines a term that contradicts that term's definition in this application, this application controls.

Definitions

The term “nucleotide base” refers to a substituted or unsubstituted aromatic ring or rings. In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases, e.g., adenine, guanine, cytosine, uracil, and thymine, and analogs of the naturally occurring nucleotide bases, e.g., 7-deazaadenine, 7-deazaguanine, 7-deaza-8-azaguanine, 7-deaza-8-azaadenine, N6-Δ2-isopentenyladenine (6iA), N6-Δ2-isopentenyl-2-methylthioadenine (2 ms6iA), N2-dimethylguanine (dmG), 7-methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT published application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide” refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT published application nos. WO 98/22489, WO 98/39352, and WO 99/14226). Exemplary LNA sugar analogs within a polynucleotide include, but are not limited to, the structures: 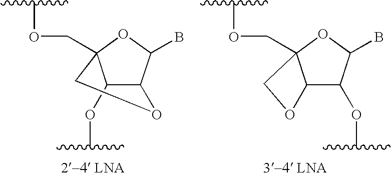
where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, amino, alkylamino, fluoro, chloro and bromo. Nucleotides include, but are not limited to, the natural D optical isomer, as well as the L optical isomer forms (see, e.g., Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleotide base is purine, e.g. A or G, the ribose sugar is attached to the N⁹-position of the nucleotide base. When the nucleotide base is pyrimidine, e.g. C, T or U, the pentose sugar is attached to the N¹-position of the nucleotide base, except for pseudouridines, in which the pentose sugar is attached to the C5 position of the uracil nucleotide base (see, e.g., Kornberg and Baker, (1992) DNA Replication, 2^{nd} Ed., Freeman, San Francisco, Calif.).

One or more of the pentose carbons of a nucleotide may be substituted with a phosphate ester having the formula: 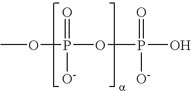
where α is an integer from 0 to 4. In certain embodiments, α is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g. α-thio-nucleotide 5′-triphosphates. For a review of nucleotide chemistry, see, e.g., Shabarova, Z. and Bogdanov, A. Advanced Organic Chemistry of Nucleic Acids, VCH, New York, 1994.

The term “nucleotide analog” refers to embodiments in which the pentose sugar and/or the nucleotide base and/or one or more of the phosphate esters of a nucleotide may be replaced with its respective analog. In certain embodiments, exemplary pentose sugar analogs are those described above. In certain embodiments, the nucleotide analogs have a nucleotide base analog as described above. In certain embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may include associated counterions.

Also included within the definition of “nucleotide analog” are nucleotide analog monomers which can be polymerized into polynucleotide analogs in which the DNA/RNA phosphate ester and/or sugar phosphate ester backbone is replaced with a different type of internucleotide linkage. Exemplary polynucleotide analogs include, but are not limited to, peptide nucleic acids, in which the sugar phosphate backbone of the polynucleotide is replaced by a peptide backbone.

An “extendable nucleotide” is a nucleotide which is: (i) capable of being enzymatically or synthetically incorporated onto the terminus of a polynucleotide chain, and (ii) capable of supporting further enzymatic or synthetic extension. Extendable nucleotides include nucleotides that have already been enzymatically or synthetically incorporated into a polynucleotide chain, and have either supported further enzymatic or synthetic extension, or are capable of supporting further enzymatic or synthetic extension. Extendable nucleotides include, but are not limited to, nucleotide 5′-triphosphates, e.g., dNTP and NTP, phosphoramidites suitable for chemical synthesis of polynucleotides, and nucleotide units in a polynucleotide chain that have already been incorporated enzymatically or chemically.

The term “nucleotide terminator” or “terminator” refers to an enzymatically-incorporable nucleotide, which does not support incorporation of subsequent nucleotides in a primer extension reaction. A terminator is therefore not an extendable nucleotide. In certain embodiments, terminators are those in which the nucleotide is a purine, a 7-deaza-purine, a pyrimidine, or a nucleotide analog, and the sugar moiety is a pentose which includes a 3′-substituent that blocks further synthesis, such as a dideoxynucleotide triphosphate (ddNTP). In certain embodiments, substituents that block further synthesis include, but are not limited to, amino, deoxy, halogen, alkoxy and aryloxy groups. Exemplary terminators include, but are not limited to, those in which the sugar-phosphate ester moiety is 3′-(C1-C6)alkylribose-5′-triphosphate, 2′-deoxy-3′-(C1-C6)alkylribose-5′-triphosphate, 2′-deoxy-3′-(C1-C6)alkoxyribose-5-triphosphate, 2′-deoxy-3′-(C5-C14)aryloxyribose-5′-triphosphate, 2′-deoxy-3′-haloribose-5′-triphosphate, 2′-deoxy-3′-aminoribose-5′-triphosphate, 2′,3′-dideoxyribose-5′-triphosphate or 2′,3′-didehydroribose-5′-triphosphate. Terminators include, but are not limited to, T terminators, including ddTTP and dUTP, which incorporate opposite an adenine, or adenine analog, in a template; A terminators, including ddATP, which incorporate opposite a thymine, uracil, or an analog of thymine or uracil, in the template; C terminators, including ddCTP, which incorporate opposite a guanine, or guanine analog, in the template; and G terminators, including ddGTP and ddITP, which incorporate opposite a cytosine, or cytosine analog, in the template.

As used herein, the terms “polynucleotide”, “oligonucleotide”, and “nucleic acid” are used interchangeably and refer to single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H⁺, NH₄⁺, trialkylammonium, Mg²⁺, Na⁺ and the like. A polynucleotide may be composed entirely of deoxyribonucleotides, entirely of ribonucleotides, or chimeric mixtures thereof. The nucleotide monomer units may comprise any of the nucleotides described herein, including, but not limited to, nucleotides and nucleotide analogs. A polynucleotide may comprise one or more lesions. Polynucleotides typically range in size from a few monomeric units, e.g. 5-40 when they are sometimes referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine or an analog thereof, “C” denotes deoxycytidine or an analog thereof, “G” denotes deoxyguanosine or an analog thereof, and “T” denotes thymidine or an analog thereof, unless otherwise noted.

Polynucleotides may be composed of a single type of sugar moiety, e.g., as in the case of RNA and DNA, or mixtures of different sugar moieties, e.g., as in the case of RNA/DNA chimeras. In certain embodiments, nucleic acids are ribopolynucleotides and 2′-deoxyribopolynucleotides according to the structural formulae below: 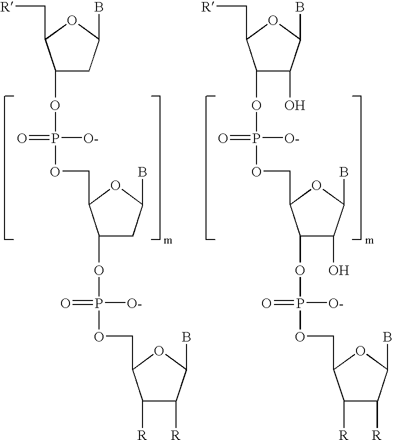
wherein each B is independently the base moiety of a nucleotide, e.g., a purine, a 7-deazapurine, a pyrimidine, or an analog thereof; each m defines the length of the respective nucleic acid and can range from zero to thousands, tens of thousands, or even more; each R is independently selected from the group comprising hydrogen, hydroxyl, halogen, —R″, —OR″, and —NR″R″, where each R″ is independently (C₁-C₆) alkyl or (C₅-C1₄) aryl, or two adjacent Rs may be taken together to form a bond such that the ribose sugar is 2′,3′-didehydroribose, and each R′ may be independently hydroxyl or 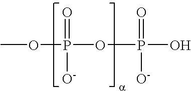
where α is zero, one or two.

In certain embodiments of the ribopolynucleotides and 2′-deoxyribopolynucleotides illustrated above, the nucleotide bases B are covalently attached to the C1′ carbon of the sugar moiety as previously described.

The terms “nucleic acid”, “polynucleotide”, and “oligonucleotide” may also include nucleic acid analogs, polynucleotide analogs, and oligonucleotide analogs. The terms “nucleic acid analog”, “polynucleotide analog” and “oligonucleotide analog” are used interchangeably, and refer to a polynucleotide that contains at least one nucleotide analog and/or at least one phosphate ester analog and/or at least one pentose sugar analog. A polynucleotide analog may comprise one or more lesions. Also included within the definition of polynucleotide analogs are polynucleotides in which the phosphate ester and/or sugar phosphate ester linkages are replaced with other types of linkages, such as N-(2-aminoethyl)-glycine amides and other amides (see, e.g., Nielsen et al., 1991, Science 254: 1497-1500; WO 92/20702; U.S. Pat. No. 5,719,262; U.S. Pat. No. 5,698,685;); morpholinos (see, e.g., U.S. Pat. No. 5,698,685; U.S. Pat. No. 5,378,841; U.S. Pat. No. 5,185,144); carbamates (see, e.g., Stirchak & Summerton, 1987, J. Org. Chem. 52: 4202); methylene(methylimino) (see, e.g., Vasseur et al., 1992, J. Am. Chem. Soc. 114: 4006); 3′-thioformacetals (see, e.g., Jones et al., 1993, J. Org. Chem. 58: 2983); sulfamates (see, e.g., U.S. Pat. No. 5,470,967); 2-aminoethylglycine, commonly referred to as PNA (see, e.g., Buchardt, WO 92/20702; Nielsen (1991) Science 254:1497-1500); and others (see, e.g., U.S. Pat. No. 5,817,781; Frier & Altman, 1997, Nucl. Acids Res. 25:4429 and the references cited therein). Phosphate ester analogs include, but are not limited to, (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate.

The term “label” refers to any molecule that can be detected. In certain embodiments, a label can be a moiety that produces a signal or that interacts with another moiety to produce a signal. In certain embodiments, a label can interact with another moiety to modify a signal of the other moiety. In certain embodiments, a label can bind to another moiety or complex that produces a signal or that interacts with another moiety to produce a signal.

Labels may be “detectably different”, which means that they are distinguishable from one another by at least one detection method. Detectably different labels include, but are not limited to, labels that emit light of different wavelengths, labels that absorb light of different wavelengths, labels that have different fluorescent decay lifetimes, labels that have different spectral signatures, labels that have different radioactive decay properties, labels of different charge, and labels of different size.

The term “labeled terminator” refers to a terminator that is physically joined to a label. The linkage to the label is at a site or sites on the terminator that do not prevent the incorporation of the terminator by a polymerase into a polynucleotide.

The term “target nucleic acid sequence” refers to a nucleic acid sequence that serves as a template for a primer extension reaction.

Different target nucleic acid sequences may be different portions of a single contiguous nucleic acid or may be on different nucleic acids. Different portions of a single contiguous nucleic acid may overlap.

A target nucleic acid sequence may comprise one or more lesions. In certain embodiments, a target nucleic acid sequence comprising one or more lesions is called a “lesion-containing target nucleic acid sequence.” Lesions include, but are not limited to, one or more nucleotides with at least one abnormal alteration in its chemical properties, e.g., a base alteration, a base deletion, a sugar alteration, or an alteration which causes a strand break. Specifically, lesions include, but are not limited to, abasic sites; AAF adducts, including, but not limited to, N-(deoxyguanosine-8-yl)-2-acetylaminofluorene and N-(deoxyguanosine-8-yl)-2-aminofluorene; cis-cyn pyrimidine dimers (also referred to as cyclobutane pyrimidine dimers), including, but not limited to, cis-syn thymine-thymine dimers; 6-4 pyrimidine-pyrimidone dimers; benzo[a]pyrene diol epoxide adducts, including, but not limited to, benzo[a]pyrene diol epoxide deoxyadenosine adducts and benzo[a]pyrene diol epoxide deoxyguanosine adducts; oxidized guanine, including, but not limited to, 7,8-dihydro-8-oxoguanine, and 8-oxoguanine, (8-hydroxyguanine); oxidized adenine, including, but not limited to, 7,8-dihydro-8-oxoadenine, and 8-oxoadenine, (8-hydroxyadenine); 5-hydroxycytosine; 5-hydroxyuracil; 5,6-dihydouracil; cisplatin adducts, including but not limited to, 1,2-cisplatinated guanine; 5,6-dihydro-5,6-dihyroxythymine (thymine glycol); 1,N⁶-ethenodeoxyadenosine; O⁶-methylguanine; cyclodeoxyadenosine; 2,6-diamino-4-hydroxyformamidopyrimidine; 8-nitroguanine; N²-guanine monoadducts of 1,3-butadiene metabolites; and oxidized cytosine.

Lesions also include, but are not limited to, any alteration in a polynucleotide resulting from radiation, oxidative damage, and chemical mutagens. Sources of radiation include, but are not limited to, nonionizing radiation (e.g., UV radiation), or ionizing radiation (e.g., X-rays, gamma radiation, and corpuscular radiation (e.g., α-particle and β-particle radiation)). Sources of oxidative damage include, but are not limited to, oxidative damage mediated by one or more transition metals (e.g., the combination of H₂O₂ and CuCl₂)), and chemical mutagens. Chemical mutagens include, but are not limited to, base analogs (e.g., bromouracil or aminopurine), chemicals which alter the structure and pairing properties of bases (e.g., nitrous acid, nitrosoguanidine, methyl methanesulfonate (MMS), and ethyl methanesulfonate (EMS)), intercalating agents (e.g., ethidium bromide, acridine orange, and proflavin), agents altering DNA structure (e.g., large molecules that bind to bases in DNA and cause them to be noncoding (e.g., acetyl aminofluorene (AAF), N-acetoxy-2-aminofluorene (NAAAF), or cisplatin), agents causing inter- and intrastrand crosslinks (e.g., psoralens), methylated and acetylated bases, and chemicals causing DNA strand breaks (e.g., peroxides)).

The term “microsatellite” refers to a repetitive stretch of a short sequence of DNA. In certain embodiments, the short sequence of DNA is two bases in length. In certain embodiments, the short sequence of DNA is three bases in length. In certain embodiments, the short sequence of DNA is four bases in length. In certain embodiments, the short sequence of DNA is more than four bases in length. In certain embodiments, microsatellites include short tandem repeats (STRs). In certain embodiments, microsatellites can be used as genetic markers.

The term “genotype” refers to the specific allelic composition of one or more genes of an organism. The term “genotyping” refers to testing that reveals certain specific alleles carried by an individual.

The terms “annealing” and “hybridization” are used interchangeably and refer to the base-pairing interaction of one nucleic acid with another nucleic acid that results in formation of a duplex, triplex, or other higher-ordered structure. In certain embodiments, the primary interaction is base specific, e.g., A/T and G/C, by Watson/Crick and Hoogsteen-type hydrogen bonding. Base-stacking and hydrophobic interactions may also contribute to duplex stability.

In this application, a statement that one sequence is the same as or is complementary to another sequence encompasses situations where both of the sequences are completely the same or complementary to one another, and situations where only a portion of one of the sequences is the same as, or is complementary to, a portion or the entire other sequence. Here, the term “sequence” encompasses, but is not limited to, nucleic acid sequences, polynucleotides, oligonucleotides, probes, primers, primer-specific portions, and target-specific portions.

In this application, a statement that one sequence is complementary to another sequence encompasses situations in which the two sequences have mismatches. Here, the term “sequence” encompasses, but is not limited to, nucleic acid sequences, polynucleotides, oligonucleotides, probes, primers, primer-specific portions, and target-specific portions. Despite the mismatches, the two sequences should selectively hybridize to one another under appropriate conditions.

The term “selectively hybridize” means that, for particular identical sequences, a substantial portion of the particular identical sequences hybridize to a given desired sequence or sequences, and a substantial portion of the particular identical sequences do not hybridize to other undesired sequences. A “substantial portion of the particular identical sequences” in each instance refers to a portion of the total number of the particular identical sequences, and it does not refer to a portion of an individual particular identical sequence. In certain embodiments, “a substantial portion of the particular identical sequences” means at least 70% of the particular identical sequences. In certain embodiments, “a substantial portion of the particular identical sequences” means at least 80% of the particular identical sequences. In certain embodiments, “a substantial portion of the particular identical sequences” means at least 90% of the particular identical sequences. In certain embodiments, “a substantial portion of the particular identical sequences” means at least 95% of the particular identical sequences.

In certain embodiments, the number of mismatches that may be present may vary in view of the complexity of the composition. Thus, in certain embodiments, the more complex the composition, the more likely undesired sequences will hybridize. For example, in certain embodiments, with a given number of mismatches, a probe may more likely hybridize to undesired sequences in a composition with the entire genomic DNA than in a composition with fewer DNA sequences, when the same hybridization and wash conditions are employed for both compositions. Thus, that given number of mismatches may be appropriate for the composition with fewer DNA sequences, but fewer mismatches may be more optimal for the composition with the entire genomic DNA.

In certain embodiments, sequences are complementary if they have no more than 20% mismatched nucleotides. In certain embodiments, sequences are complementary if they have no more than 15% mismatched nucleotides. In certain embodiments, sequences are complementary if they have no more than 10% mismatched nucleotides. In certain embodiments, sequences are complementary if they have no more than 5% mismatched nucleotides.

In this application, a statement that one sequence hybridizes or binds to another sequence encompasses situations where the entirety of both of the sequences hybridize or bind to one another, and situations where only a portion of one or both of the sequences hybridizes or binds to the entire other sequence or to a portion of the other sequence. Here, the term “sequence” encompasses, but is not limited to, nucleic acid sequences, polynucleotides, oligonucleotides, probes, primers, primer-specific portions, and target-specific portions.

A “probe” is an polynucleotide that is capable of binding to a complementary target sequence.

The term “primer” refers to a polynucleotide that has a free 3′-OH (or functional equivalent thereof) that can be extended by at least one nucleotide in a primer extension reaction catalyzed by a polymerase. In certain embodiments, primers may be of virtually any length, provided they are sufficiently long to hybridize to a polynucleotide of interest in the environment in which primer extension is to take place. Primers may be specific for a particular sequence, or, alternatively, may be degenerate, e.g., specific for a set of sequences.

The terms “primer extension” and “primer extension reaction” are used interchangeably, and refer to a process of adding one or more nucleotides to a nucleic acid primer, or to a primer extension product, using a polymerase, a template, and one or more nucleotides.

A “primer extension product” is produced when one or more nucleotides have been added to a primer, or to a primer extension product, in a primer extension reaction. In certain embodiments, a primer extension product serves as a target nucleic acid sequence in subsequent primer extension reactions. In certain embodiments, a primer extension product includes a terminator. In certain embodiments, when a primer extension product includes a terminator, it is referred to as a “primer extension product comprising a terminator.”

The terms “primer set” or “set of primers” refer to two or more primers that are used as a set. In certain embodiments, a primer set comprises hundreds of different primers. In certain such embodiments, the genus of primers of a primer set may be represented by a formula, e.g., CTNNNNNNNN.

When used to describe primer sets, the following symbols have the following meanings:

N: A random nucleotide. This can be a natural or non-natural nucleotide.

A: Adenine

T: Thymine

G: Guanine

C: Cytosine

U: Uracil

I: Inosine

t: 3′ phosphorothioates

5: 5′ nitroindoles

Y: Cytosine or Thymidine/Uracil

R: Guanine or Adenine

M: Adenine or Cytosine

W: Adenine or Thymine or Uracil

S: Guanine or Cytosine

K: Guanine or Thymine or Uracil

B: Guanine or Cytosine or Thymine or Uracil

D: Adenine or Guanine or Thymine or Uracil

H: Adenine or Cytosine or Thymine or Uracil

V: Adenine or Guanine or Cytosine

The term “polypeptide” is used herein as a generic term to refer to any polypeptide comprising two or more amino acids joined to each other by peptide bonds or modified peptide bonds. The term “polypeptide” encompasses polypeptides regardless of length or origin, comprising molecules that are recombinantly produced or naturally occurring, full length or truncated, having a natural sequence or mutated sequence, with or without post-translational modification, whether chemically synthesized or produced in mammalian cells, bacterial cells, or any other expression system. In certain embodiments, polypeptides are randomly generated. In certain embodiments, shorter polypeptides are derived by digestion of larger polypeptides.

The term “variant” refers to any alteration of a polypeptide, including, but not limited to, changes in amino acid sequence, substitutions of one or more amino acids, addition of one or more amino acids, deletion of one or more amino acids, and alterations to the amino acids themselves. In certain embodiments, the changes involve conservative amino acid substitutions. Conservative amino acid substitution may involve replacing one amino acid with another that has, e.g., similar hydrophobicity, hydrophilicity, charge, or aromaticity. In certain embodiments, conservative amino acid substitutions may be made on the basis of similar hydropathic indices. A hydropathic index takes into account the hydrophobicity and charge characteristics of an amino acid, and, in certain embodiments, may be used as a guide for selecting conservative amino acid substitutions. The hydropathic index is discussed, e.g., in Kyte et al., J. Mol. Biol., 157:105-131 (1982). It is understood in the art that conservative amino acid substitutions may be made on the basis of any of the aforementioned characteristics.

The term “polymerase” refers to an enzyme that is capable of adding at least one nucleotide onto the 3′ end of a primer, or to a primer extension product, that is annealed to a target nucleic acid sequence. In certain embodiments, the nucleotide is added to the 3′ end of the primer in a template-directed manner. In certain embodiments, the polymerase is capable of sequentially adding two or more nucleotides onto the 3′ end of the primer. In certain embodiments, the polymerase is active at 37° C. In certain embodiments, the polymerase is active at a temperature other than 37° C. In certain embodiments, the polymerase is active at a temperature greater than 37° C. In certain embodiments, the polymerase is active at both 37° C. and other temperatures. A “DNA polymerase” catalyzes the polymerization of deoxynucleotides.

The term “thermostable polymerase” refers to a polymerase that retains its ability to add at least one nucleotide onto the 3′ end of a primer, or to a primer extension product, that is annealed to a target nucleic acid sequence at a temperature higher than 37° C. In certain embodiments, the thermostable polymerase remains active at a temperature greater than about 37° C. In certain embodiments, the thermostable polymerase remains active at a temperature greater than about 42° C. In certain embodiments, the thermostable polymerase remains active at a temperature greater than about 50° C. In certain embodiments, the thermostable polymerase remains active at a temperature greater than about 60° C. In certain embodiments, the thermostable polymerase remains active at a temperature greater than about 70° C. In certain embodiments, the thermostable polymerase remains active at a temperature greater than about 80° C. In certain embodiments, the thermostable polymerase remains active at a temperature greater than about 90° C. The term “non-thermostable polymerase” refers to a polymerase that does not retain its ability to add at least one nucleotide onto the 3′ end of a primer, or to a primer extension product, that is annealed to a target nucleic acid sequence at a temperature higher than 37° C.

In certain embodiments, a polymerase is a processive polymerase. In certain embodiments, a processive polymerase remains associated with the template for two or more nucleotide additions. In certain embodiments, a non-processive polymerase disassociates from the template after the addition of each nucleotide. In certain embodiments, a processive DNA polymerase has a characteristic polymerization rate. In certain embodiments, a processive DNA polymerase has a polymerization rate of between 5 to 300 nucleotides per second. In certain embodiments, a processive DNA polymerase has a higher processivity in the presence of accessory factors. For example, and not limitation, the processivity of a processive DNA polymerase may be influenced by the presence or absence of accessory ssDNA binding proteins and helicases. In certain embodiments, where the polymerase is a non-processive polymerase, the net polymerization rate will depend on the enzyme concentration, because at higher concentrations there are more re-initiation events and thus the net polymerization rate is increased.

In certain embodiments, a DNA polymerase is a strand displacement polymerase. In certain embodiments, a processive DNA polymerase is also a strand displacement polymerase. A strand displacement polymerase is capable of displacing a hybridized strand encountered during replication. In certain embodiments, a strand displacement polymerase requires a strand displacement factor to be capable of displacing a hybridized strand encountered during replication. A “strand displacement factor” is a factor that facilitates strand displacement. In certain embodiments, a strand displacement polymerase is capable of displacing a hybridized strand encountered during replication in the absence of a strand displacement factor. In certain embodiments, the strand displacement polymerase lacks 5′ to 3′ exonuclease activity.

“Strand displacement” as used herein refers to the phenomenon in which a chemical, physical, or biological agent causes at least partial dissociation of a nucleic acid that is hybridized to its complementary strand. In certain embodiments, the dissociation of a nucleic acid that is hybridized to its complementary strand occurs in a 5′ to 3′ direction in conjunction with replication. In certain embodiments, where a primer extension reaction forms a newly synthesized strand while displacing a second nucleic acid strand from the template nucleic acid strand, both the newly synthesized and displaced second nucleic acid strand have the same base sequence, which is complementary to the template nucleic acid strand. In certain embodiments, a molecule comprises both strand displacement activity and another activity. For example, and not limitation, in certain embodiments, a molecule comprises both strand displacement activity and polymerase activity. In certain embodiments, strand displacement activity is the only activity associated with a molecule. Enzymes which possess both strand displacement activity and polymerase activity include, but are not limited to, E. coli DNA polymerase I, the Klenow fragment of DNA polymerase I, the bacteriophage T7 DNA polymerase, the bacteriophage T5 DNA polymerase, the φ29 polymerase, and the Bst polymerase. Certain methods of using enzymes possessing strand displacement activity are known in the art. See, e.g., Kornberg, A., DNA Replication, W.H. Freeman & Co., San Francisco, Calif., 1980.

The term “strand displacement replication” refers to nucleic acid replication which involves strand displacement. In certain embodiments, strand displacement is facilitated through the use of a strand displacement factor, such as helicase. In certain embodiments, a DNA polymerase that can perform strand displacement replication in the presence of a strand displacement factor is used in strand displacement replication. In certain such embodiments, the DNA polymerase does not perform strand displacement replication in the absence of such a factor. Exemplary strand displacement factors useful in strand displacement replication include, but are not limited to, BMRF1 polymerase accessory subunit (Tsurumi et al., J. Virology 67(12):7648-7653 (1993)), adenovirus DNA-binding protein (Zijderveld and van der Vliet, J. Virology 68(2):1158-1164 (1994)), herpes simplex viral protein ICP8 (Boehmer and Lehnan, J. Virology 67(2):711-715 (1993); Skaliter and Lehman, Proc. Natl. Acad. Sci. USA 91(22):10665-10669 (1994)); single-stranded DNA binding proteins (SSB; Rigler and Romano, J. Biol. Chem. 270:8910-8919 (1995)); phage T4 gene 32 protein (Villemain and Giedroc, Biochemistry 35:14395-14404 (1996); and calf thymus helicase (Siegel et al., J. Biol. Chem. 267:13629-13635 (1992)).

In certain instances, the ability of a polymerase to carry out strand displacement replication can be determined by using the polymerase in a strand displacement replication assay such as those described, e.g., in U.S. Pat. No. 6,642,034. Another exemplary assay for selecting a strand displacement polymerase is the primer-block assay described, e.g., in Kong et al., J. Biol. Chem. 268:1965-1975 (1993). Such assays are primer extension assays that use an M13 ssDNA template in the presence or absence of an oligonucleotide that is hybridized upstream of the extending primer to block its progress. Enzymes that are able to displace the blocking primer in such an assay are capable of strand displacement.

In certain embodiments, a processive polymerase is used in an isothermal amplification reaction, such as strand displacement amplification (SDA). SDA is described, e.g., in Fraiser et al., U.S. Pat. No. 5,648,211; Cleuziat et al., U.S. Pat. No. 5,824,517; and Walker et al., Proc. Natl. Acad. Sci. U.S.A. 89:392-396 (1992).

The term “unit” of polymerase is defined as the amount of polymerase that will catalyze the incorporation of 10 nmoles of nucleotide into Trichloroacetic acid-insoluble material in 30 minutes. In certain embodiments, a unit of thermostable polymerase is defined at 74° C. In certain embodiments, a unit of thermostable polymerase is defined at 50° C. In certain embodiments, a unit of non-thermostable polymerase is defined at 37° C. In certain embodiments, units of polymerase are defined for specific reaction conditions.

In certain embodiments, the “unit ratio” of one polymerase to another polymerase in a composition is based on the percentage of the total units of each polymerase in the composition. For example, and not limitation, if the unit ratio of polymerase A to polymerase B is 70:30, and there are 10 total units of polymerase in the composition, then there are 7 units of polymerase A and 3 units of polymerase B. In certain embodiments, units of polymerase for two or more different polymerases are defined under the same conditions. In certain embodiments, units of polymerase for a polymerase are calculated under different conditions than the conditions used to calculate the units of polymerase for one or more other different polymerases.

In certain embodiments, the “weight ratio” of one polymerase to another polymerase in a composition is based on the percentage of the total weight of polymerases in the composition. For example and not limitation, if the weight ratio of polymerase A to polymerase B is 6:94, and there are 100 ng total polymerase in the composition, then there is 6 ng of polymerase A and 94 ng of polymerase B.

As used herein, a “buffering agent” is a compound added to a composition which modifies the stability, activity, or longevity of one or more components of the composition by regulating the pH of the composition. Buffering agents are well known in the art and include, but are not limited to, Tris and Tricine.

As used herein, an “additive” is a compound added to a composition which modifies the stability, activity, or longevity of one or more components of the composition. In certain embodiments, an additive inactivates contaminant enzymes, stabilizes protein folding, and/or decreases aggregation. Exemplary additives include, but are not limited to, glycerol, DMSO, dithiothreitol (DTT), Thermoplasma acidophilum inorganic pyrophosphatase (TAP), betaine, and bovine serum albumin (BSA).

The term “amplification bias” refers to the efficiency with which a primer set amplifies certain nucleic acids compared to certain other different nucleic acids. In certain instances, individual target nucleic acid sequences of a plurality of different target nucleic acid sequences amplified by a primer set will not be amplified equally. In other words, in certain instances, amplification of certain target nucleic acid sequences will be favored over amplification of certain other different target nucleic acid sequences. Thus, in certain such instances, some amplification products from certain target nucleic acid sequences will be more abundant than others after amplification of the target nucleic acid sequences. In certain such instances, the difference in quantity between the different amplification products is the result of amplification bias. For example and not limitation, in certain instances, a primer set will preferentially amplify smaller nucleic acids compared to longer nucleic acids. In certain instances, nucleic acids comprising GC-rich regions are amplified less than the rest of the nucleic acid sequences. In certain instances, nucleic acids comprising centromere regions are amplified less than the rest of the nucleic acid sequences. In certain instances, nucleic acids comprising telomere regions are amplified less than the rest of the nucleic acid sequences. In certain instances, nucleic acids comprising secondary structures are amplified less than the rest of the nucleic acid sequences.

In certain embodiments, the composition of the primer set affects the amplification bias. Thus, in certain embodiments, different primer sets, with different sequences will have different amplification biases.

In certain embodiments, differences between amplification biases between different primer sets can be seen by examining the amplification profiles of the different primer sets. The term “amplification profile” refers to the results of an analysis of amplification products produced by a set of primers. In certain embodiments, an amplification profile can be determined by quantitating the amplification products comprising a portion or portions of a nucleic acid. In certain embodiments, an amplification profile is determined by quantitating the amplification products comprising thirty or more portions.

For example and not limitation, where a first primer set and a second primer set are used to amplify the same plurality of target nucleic acid sequences under the same conditions, the first primer set may produce more amplification product comprising a first portion than the second primer set. That second primer set may, however, produce more amplification product comprising a second portion than the first primer set. Thus, the first primer set has a different amplification profile from the second primer set. In certain embodiments, a third primer set may produce more amplification product comprising the first portion and amplification product comprising the second portion than either the first primer set or the second primer set. That third primer set would have a different amplification profile than either the first primer set or the second primer set. In certain embodiments, each primer set has a distinct amplification profile.

In certain embodiments, one incorporates specific sequences into a primer set to change the amplification profile. For example, and not limitation, the primer set with the sequence N₁₁ poorly amplifies nucleic acid targets comprising telomere regions. However, if one changes the primer set to a primer set with the sequence N₄T₃N₄, the amplification of nucleic acid targets comprising telomere regions is improved. Thus, the amplification profile changes.

In certain embodiments, a primer comprises a random portion. The term “random portion,” when used to refer to a primer, refers to a portion where each position in that portion can comprise any nucleotide and no nucleotides are intentionally excluded from that portion. The possible nucleotides in a random portion always comprise either (i) (a) A or an analog of A, (b) G or an analog of G, (c) C or an analog of C, and (d) T or an analog of T, or (ii) (a) A or an analog of A, (b) G or an analog of G, (c) C or an analog of C, and (d) U or an analog of U. In certain embodiments, the possible nucleotides in a random portion also comprise nucleotides other than A or an analog of A, G or an analog of G, C or an analog of C, T or an analog of T, and U or an analog of U. A nucleotide in a random portion may be any naturally occurring or non-naturally occurring nucleotide. In certain embodiments, a random portion consists entirely of random nucleotides selected from A, G, C, and T.

In certain embodiments, a primer is a random primer. The term “random primer” refers to a primer which consists of a random portion.

Where a primer is represented as a formula, the “N” in the formula represents a random nucleotide. For example, in the primer set represented by TTTTN, the N represents a random nucleotide. Because the random nucleotide represents many different nucleotides, the primer set may include, but is not limited to, TTTTT, TTTTA, TTTTG, and TTTTC. If non-natural nucleotides were used during the synthesis of the primer, the primer set may include primers with those non-natural nucleotides incorporated into the primer at position “N” as well as the primers listed above.

The term “designed portion,” when used to refer to a primer, refers to a portion of a primer where each position in that portion excludes the possibility of one or more nucleotides. In addition, where a primer set comprises a designed portion and a random portion, the designed portion causes the primer set to have a different amplification profile when used to amplify target nucleic acid sequences than a primer set of the same length consisting entirely of random primers. For example and not limitation, the primer set corresponding to the sequence CTN₈ has a non-random portion represented by the CT, and a different amplification profile than the primer set corresponding to the sequence N₁₀. Thus, the portion of the primer set corresponding to the sequence CT is a designed portion.

In certain embodiments, a designed portion includes some variation. For example, in certain embodiments, a primer set is represented by RN₈, where the designed portion is R, which is either Guanine or Adenine. Thus, although the primer set represented by RN₈ has eight random positions, the designed portion, R, of that primer is not random, because only G or A can be present, and all other nucleotides are excluded. Therefore, the R is not a random portion. Another example of a primer set that includes a designed portion that comprises a subset of all the possible nucleotides is SSNNNNNNNN, where S represents either G or C. Thus, in certain such embodiments, the primer set represented by SSNNNNNNNN would not include primers having an A or T at the first two positions, and, in certain embodiments, would comprise the primers GGNNNNNNNN, GCNNNNNNNN, CGNNNNNNNN, and CCNNNNNNNN. In certain embodiments, the nucleotides in a designed portion are non-natural nucleotides.

In certain embodiments, a primer set includes a constant portion. The term “constant portion” refers to a portion that consists of nucleotides with no variation. In certain embodiments, a designed portion comprises a constant portion. For example, and not limitation, the primer set represented by the formula CTN₈ includes a constant portion represented by the CT. As discussed above, that portion is also a designed portion.

Certain Exemplary Components

In various embodiments, a label is attached to a molecule and: (i) provides a detectable signal; (ii) interacts with a second label to modify the detectable signal provided by the second label, e.g., FRET (Fluorescent Resonance Energy Transfer); (iii) stabilizes hybridization, e.g., duplex formation; or (iv) provides a member of a binding complex or affinity set, e.g., affinity, antibody/antigen, ionic complexes, hapten/ligand, e.g., biotin/avidin.

In various embodiments, use of labels can be accomplished using any one of a large number of known techniques employing known labels, linkages, linking groups, reagents, reaction conditions, and analysis and purification methods. Labels include, but are not limited to, light-emitting or light-absorbing compounds which generate or quench a detectable fluorescent, chemiluminescent, or bioluminescent signal (see, e.g., Kricka, L. in Nonisotopic DNA Probe Techniques (1992), Academic Press, San Diego, pp. 3-28). Fluorescent reporter dyes useful as labels include, but are not limited to, fluoresceins (see, e.g., U.S. Pat. Nos. 5,188,934; 6,008,379; and 6,020,481), rhodamines (see, e.g., U.S. Pat. Nos. 5,366,860; 5,847,162; 5,936,087; 6,051,719; and 6,191,278), benzophenoxazines (see, e.g., U.S. Pat. No. 6,140,500), energy-transfer fluorescent dyes, comprising pairs of donors and acceptors (see, e.g., U.S. Pat. Nos. 5,863,727; 5,800,996; and 5,945,526), and cyanines (see, e.g., Kubista, WO 97/45539), as well as any other fluorescent moiety capable of generating a detectable signal. Examples of fluorescein dyes include, but are not limited to, 6-carboxyfluorescein; 2′,4′,1,4,-tetrachlorofluorescein; and 2′,4′,5′,7′,1,4-hexachlorofluorescein.

A class of labels are hybridization-stabilizing moieties which serve to enhance, stabilize, or influence hybridization of duplexes, e.g. intercalators and intercalating dyes (including, but not limited to, ethidium bromide and cyber green), minor-groove binders, and cross-linking functional groups (see, e.g., Blackburn, G. and Gait, M. Eds. “DNA and RNA structure” in Nucleic Acids in Chemistry and Biology, 2^{nd} Edition, (1996) Oxford University Press, pp. 15-81). Yet another class of labels effect the separation or immobilization of a molecule by specific or non-specific capture, for example biotin, digoxigenin, and other haptens (see, e.g., Andrus, A. “Chemical methods for 5′ non-isotopic labeling of PCR probes and primers” (1995) in PCR 2: A Practical Approach, Oxford University Press, Oxford, pp. 39-54). Non-radioactive labelling methods, techniques, and reagents are reviewed in: Non-Radioactive Labelling, A Practical Introduction, Garman, A. J. (1997) Academic Press, San Diego.

In certain embodiments, target nucleic acid sequences include RNA and DNA. Exemplary RNA target sequences include, but are not limited to, mRNA, rRNA, tRNA, snRNA, viral RNA, and variants of RNA, such as splicing variants. Exemplary DNA target sequences include, but are not limited to, genomic DNA, plasmid DNA, phage DNA, nucleolar DNA, mitochondrial DNA, chloroplast DNA, cDNA., synthetic DNA, yeast artificial chromosomal DNA (“YAC”), bacterial artificial chromosome DNA (“BAC”), other extrachromosomal DNA, and primer extension products. Target nucleic acid sequences also include, but are not limited to, analogs of both RNA and DNA. Exemplary nucleic acid analogs include, but are not limited to, locked nucleic acids (“LNAs”), peptide nucleic acids (“PNAs”), 8-aza-7-deazaguanine (“PPG's”), and other nucleic acid analogs. In certain embodiments, target nucleic acid sequences include chimeras of RNA and DNA.

A variety of methods are available for obtaining a target nucleic acid sequence for use with the compositions and methods of the present invention. When the nucleic acid target is obtained through isolation from a biological matrix, certain isolation techniques include, but are not limited to, (1) organic extraction followed by ethanol precipitation, e.g., using a phenol/chloroform organic reagent (e.g., Ausubel et al., eds., Current Protocols in Molecular Biology Volume 1, Chapter 2, Section I, John Wiley & Sons, New York (1993)), in certain embodiments, using an automated DNA extractor, e.g., the Model 341 DNA Extractor available from Applied Biosystems (Foster City, Calif.); (2) stationary phase adsorption methods (e.g., Boom et al., U.S. Pat. No. 5,234,809; Walsh et al., Biotechniques 10(4): 506-513 (1991)); and (3) salt-induced DNA precipitation methods (e.g., Miller et al., Nucleic Acids Research, 16(3): 9-10 (1988)), such precipitation methods being typically referred to as “salting-out” methods. In certain embodiments, the above isolation methods may be preceded by an enzyme digestion step to help eliminate unwanted protein from the sample, e.g., digestion with proteinase K, or other like proteases. See, e.g., U.S. patent application Ser. No. 09/724,613.

In certain embodiments, a target nucleic acid sequence may be derived from any living, or once living, organism, including but not limited to prokaryote, eukaryote, plant, animal, and virus. In certain embodiments, the target nucleic acid sequence may originate from a nucleus of a cell, e.g., genomic DNA, or may be extranuclear nucleic acid, e.g., plasmid, mitrochondrial nucleic acid, various RNAs, and the like. In certain embodiments, if the sequence from the organism is RNA, it may be reverse-transcribed into a cDNA target nucleic acid sequence. Furthermore, in certain embodiments, the target nucleic acid sequence may be present in a double stranded or single stranded form.

Exemplary target nucleic acid sequences include, but are not limited to, amplification products, ligation products, transcription products, reverse transcription products, primer extension products, methylated DNA, and cleavage products. Exemplary amplification products include, but are not limited to, PCR and isothermal products.

In certain embodiments, nucleic acids in a sample may be subjected to a cleavage procedure. In certain embodiments, such cleavage products may be targets.

Different target nucleic acid sequences may be different portions of a single contiguous nucleic acid or may be on different nucleic acids. Different portions of a single contiguous nucleic acid may or may not overlap.

In certain embodiments, a target nucleic acid sequence may be derived from a crude cell lysate. Examples of target nucleic acid sequences include, but are not limited to, nucleic acids from buccal swabs, crude bacterial lysates, blood, skin, semen, hair, bone, urine, feces, nasal secretions, food products, fingerprints, filtered organisms from air filtration, and filtered organisms from consumer goods industrial production facilities.

In certain embodiments, a target nucleic acid sequence may comprise one or more forensic markers. The term “forensic marker” refers to one or more characteristics which can be used to distinguish a first nucleic acid from a second nucleic acid. In certain embodiments, one or more forensic markers can be used to distinguish the source of a first nucleic acid from the source of a second nucleic acid. In certain embodiments, a single forensic marker can be used to distinguish the source of a first nucleic acid from the source of a second nucleic acid. In certain embodiments, two or more forensic markers can be used to distinguish the source of a first nucleic acid from the source of a second nucleic acid.

In certain embodiments, a probe may include Watson-Crick bases or modified bases. Modified bases include, but are not limited to, the AEGIS bases (from Eragen Biosciences), which have been described, e.g., in U.S. Pat. Nos. 5,432,272; 5,965,364; and 6,001,983. Additionally, bases may be joined by a natural phosphodiester bond or a different chemical linkage. Different chemical linkages include, but are not limited to, a peptide bond or a Locked Nucleic Acid (LNA) linkage, which is described, e.g., in published PCT applications WO 00/56748; and WO 00/66604.

Certain Exemplary Methods of Amplification

Certain high-throughput assays that characterize multiple nucleic acid sequences from genomes are used for genetic analysis. Certain current assays can analyze genotypes at the level of thousands of single nucleotide polymorphisms (SNPs) per sample. An important aspect of certain of these techniques is generating sufficient nucleic acid for the assays. In certain cases, for example, where the sample is limited, the lack of sufficient nucleic acid can negatively impact the usefulness of the assays.

Amplification of genomic DNA solves certain sample limitation issues. However, certain available techniques to amplify genomic DNA fail to amplify the target nucleic acid sequences in an even manner, generating a product with a bias in certain sequences. Certain available methods include, but are not limited to, Random PCR or Primer Extension Preamplification-PCR (PEP-PCR) (Zhang et al., Proc. Natl. Acad. Sci., USA 89: 5847-51 (1992)), Linker Adapter PCR (Miyashita et al., Cytogenet. Cell Genet. 66(1): 54-57 (1994)), Tagged-PCR (Grothues et. al., Nuc. Acids Res. 21(5) 1321-1322 (1993)), Inter-Alu-PCR (Bicknell et. al., Genomics 10:186-192 (1991)), Degenerate Oligonucleotide Primed-PCR (DOP-PCR)(Cheung et al., Proc. Natl. Acad. Sci., USA 93:14676-14679 (1996)), Improved-Primer Extension Preamplification PCR (I-PEP-PCR)(Dietmaier et al., Amer. J. Pathology 154(1): 83-95 (1999) and U.S. Pat. No. 6,365,375), LL-DOP PCR (Kittler et al., Anal. Biochem. 300:237-244 (2002)), Balanced PCR amplification (Makrigiorgos et. al., Nature Biotech. 20:936-939 (2002)), Multiple Displacement Amplification (MDA) (U.S. Pat. Nos. 6,124,120 and 6,280,949), and Random Primer Amplification (RPA)(U.S. Pat. No. 5,043,272).

In certain embodiments, the methods, compositions, and kits described in this application may be used for amplification. In certain embodiments, the methods, compositions, and kits need not amplify all of the nucleic acids of a genome or all sequences in a sample.

In certain embodiments, a reaction composition is formed comprising (a) a plurality of target nucleic acid sequences, (b) at least one set of primers, and (c) at least one polymerase. In certain embodiments, the at least one set of primers comprises at least one set of primers which comprises at least one designed portion and at least one random portion.

In certain such embodiments, the reaction composition further comprises dNTPs and at least one buffering agent. In certain such embodiments, the at least one polymerase is at least one processive polymerase. In certain such embodiments, the amplification reaction is incubated under conditions that allow the formation of one or more amplification products. In certain embodiments, no strand displacement factors are required for strand displacement.

In certain embodiments, a primer is between 5 nucleotides and 35 nucleotides in length. In certain embodiments, a primer is greater than 35 nucleotides in length. In certain embodiments, a primer is less than 5 nucleotides in length. In certain embodiments, a primer is 10 nucleotides in length.

In certain embodiments, the designed portion of a primer set is at the 5′ end of the primers. In certain embodiments, the designed portion of a primer set is at the 3′ end of the primers. In certain embodiments, the designed portion of a primer set is in the center of the primers. In certain embodiments, the designed portion of a primer set includes two or more designed portions. In certain embodiments, the designed portions of a primer set are located in two or more portions separated by random portions, e.g., CGNNNSSSNN.

In certain embodiments, where a primer set comprises a designed portion, the nucleotides of the designed portion are pyrimidines. In certain embodiments, where a primer set comprises a designed portion, the nucleotides of the designed portion are purines. In certain embodiments, a reaction composition comprises a primer set comprising a random portion of at least eight random nucleotides. In certain embodiments, a reaction composition comprises a primer set comprising primers represented by the sequence CTN₈. In certain embodiments, a reaction composition comprises a primer set comprising primers represented by the sequence GAN₈. In certain embodiments, a reaction composition comprises a primer set comprising primers represented by the sequence SSN₈. In certain embodiments, a designed portion consists of two nucleotides at the 5′ end of the primer. In certain embodiments, a designed portion consists of two nucleotides at the 3′ end of the primer.

In certain embodiments, a reaction composition comprises two or more sets of primers. For example, and not limitation, in certain embodiments, a reaction composition may comprise the primers SSNNNNNNNN and NNNNTTTNNNN. In that example, the amplification reaction would comprise two primer sets. In certain embodiments, two or more primer sets with different amplification profiles may be combined such that the combination of primer sets has a third, different amplification profile. In certain such embodiments, one primer set may preferentially amplify sequences that are not well amplified by the other primer set.

In certain embodiments, the products of two or more amplification reactions may be combined. In certain such embodiments, the products of one amplification reaction may have a different amplification profile than the products of the second amplification reaction. In certain embodiments, the products of two or more amplification reactions may be combined to generate a pool of amplification products with substantially less amplification bias than any of the products of amplification reactions alone.

In certain embodiments, the method comprises a processive polymerase. In certain such embodiments, the polymerase is Bst polymerase. In certain embodiments, an amplification reaction comprises a blend of polymerases. In certain such embodiments, at least one polymerase possesses exonuclease activity. In certain embodiments, none of the polymerases in an amplification reaction possess exonuclease activity. Exemplary polymerases that may be used in an amplification reaction include, but are not limited to, φ29 DNA polymerase, taq polymerase, stoffel fragment, Bst DNA polymerase. E. coli DNA polymerase I, the Klenow fragment of DNA polymerase I, the bacteriophage T7 DNA polymerase, the bacteriophage T5 DNA polymerase, and other polymerases known in the art. In certain embodiments, a polymerase is inactive in the reaction composition and is subsequently activated at a given temperature.

In certain embodiments, the temperature of the amplification reaction is kept at isothermal reaction conditions. The term “isothermal reaction conditions” refers to conditions wherein the temperature is kept substantially constant. In certain embodiments, isothermal reaction conditions prevent the template DNA from being completely disassociated. In certain embodiments, short primers can hybridize to a double stranded template maintained at an isothermal temperature. In certain such embodiments, the primers that hybridize to the template DNA can be extended by a strand-displacing DNA polymerase. In certain embodiments, an amplification process is isothermal at 50° C. and uses Bst DNA polymerase for strand displacement and extension. In certain embodiments, an amplification process uses a fragment of Bst DNA polymerase with the 3′→5′ exonuclease activity removed (“the large fragment of Bst DNA polymerase”).

In certain embodiments, a reaction composition comprises strand displacement factors. Exemplary strand displacement factors include, but are not limited to, helicases and single stranded DNA binding protein. In certain embodiments, the temperature of the reaction affects strand displacement. In certain embodiments, a temperature of approximately 40° C., 45° C., 50° C., 55° C., 60° C., 65° C., 70° C., 75° C., 80° C., 85° C., or 90° C. facilitates strand displacement by allowing segments of double stranded DNA to separate and reanneal.

In certain embodiments, a reaction composition includes additives. Exemplary additives that may be included in an amplification reaction include, but are not limited to, betaine, formamide, KCl, CaCl₂, MgOAc, MgCl₂, NaCl, NH₄OAc, NaI, Na(CO₃)₂, LiCl, MnOAc, NMP, Trehalose, DMSO, Glycerol, Ethylene Glycol, Propylene Glycol, Glycinamide, CHES, Percoll, Aurintricarboxylic acid, Tween-20, Tween 21, Tween 40, Tween 60, Tween 85, Brij 30, NP-40, Triton X-100, CHAPS, CHAPSO, Mackernium, LDAO, Zwittergent 3-10, Zwittergent 3-14, Zwittergent SB 3-16, Empigen, NDSB-20, pyroPOase, T4G32, E. coli SSB, RecA, nicking endonucleases, 7-deazaG, dUTP and UNG, anionic detergents, cationic detergents, non-ionic detergents, zwittergent, sterol, osmolytes, cations, and any other chemical, protein, or cofactor that may alter the efficiency of amplification. In certain embodiments, two or more additives are included in an amplification reaction.

In certain embodiments, target nucleic acid sequences are first treated with a modifying agent before being used in an amplification reaction. The term “modifying agent” refers to any agent that can modify a nucleic acid.

In certain embodiments, the target nucleic acid sequence is first incubated with an agent that selectively modifies cytosine, depending on the methylation state of each cytosine. The term “selectively modifies” means that modification of cytosines occurs to a measurably lesser extent with cytosines that do not have the appropriate methylation state than with cytosines that have the appropriate methylation state. In certain embodiments, modification only occurs with target nucleic acid sequences that have a cytosine in the appropriate methylation state. In certain embodiments, a modifying agent selectively binds to methylated cytosines. In certain other embodiments, a modifying agent selectively binds to unmethylated cytosines. The term “selectively binds” means that binding of cytosines occurs to a measurably lesser extent with cytosines that do not have the appropriate methylation state than with cytosines that have the appropriate methylation state. In certain embodiments, binding only occurs in the presence of target nucleic acid sequences that have a cytosine in the appropriate methylation state.

In certain embodiments, the modifying agent selectively, chemically alters a cytosine, depending on the methylation state of the cytosine. The term “selectively, chemically alters” means that chemical alteration of cytosines occurs to a measurably lesser extent with cytosines that do not have the appropriate methylation state than with cytosines that have the appropriate methylation state. In certain embodiments, chemical alteration only occurs in the presence of target nucleic acid sequences that have a cytosine in the appropriate methylation state. In certain embodiments, a modifying agent selectively, chemically alters methylated cytosines. In certain other embodiments, a modifying agent selectively, chemically alters unmethylated cytosines.

In certain embodiments, the modifying agent selectively converts a cytosine to a converted nucleotide, depending on the methylation state of the cytosine. The term “selectively converts” means that conversion of cytosines occurs to a measurably lesser extent with cytosines that do not have the appropriate methylation state than with cytosines that have the appropriate methylation state. In certain embodiments, conversion only occurs in the presence of target nucleic acid sequences that have a cytosine in the appropriate methylation state. In certain embodiments, a modifying agent selectively converts methylated cytosines to converted nucleotides. In certain embodiments, a modifying agent selectively converts unmethylated cytosines to converted nucleotides.

In certain embodiments, bisulfite is employed as a modifying agent. See, e.g., U.S. Pat. No. 6,265,171; U.S. Pat. No. 6,331,393. Incubating target nucleic acid sequence with bisulfite results in deamination of a substantial portion of unmethylated cytosines, which converts such cytosines to uracil. Methylated cytosines are deaminated to a measurably lesser extent. In certain embodiments, the sample is then amplified or replicated, resulting in the uracil bases being replaced with thymine. Thus, in certain embodiments, a substantial portion of unmethylated cytosines ultimately become thymines, while a substantial portion of methylated cytosines remain cytosines.

In certain embodiments, other modifying agents may be used. In certain embodiments, the modifying agent need not catalyze deamination reactions and the converted nucleotide need not be uracil or thymine. Certain embodiments may employ any agent that is capable of selectively converting either methylated cytosines or unmethylated cytosines to another nucleotide.

In various embodiments, amplification products can be used for any purpose for which nucleic acids are used. For example, and not limitation, amplification products, such as whole genome amplification products, can be used for forensic purposes, for genotyping, for sequencing, for detecting SNPs, for detecting microsatellite DNA, for detecting expression of genes, for nucleic acid library construction, for detecting biowarfare agents, for detecting genetically modified food, for diagnostics applications including detecting viruses, mycoplasma, fungi, bacteria and parasitic organisms, and for any other purpose that involves manipulating or detecting nucleic acids or nucleic acid sequences.

In certain embodiments, amplification products may be used in any process that uses nucleic acids. Exemplary assays in which amplification products may be used include, but are not limited to, agarose gel electrophoresis, picogreen assays, oligonucleotide ligation assays, TaqMan assays, SNPlex assays, and assays described in U.S. Pat. Nos. 5,470,705, 5,514,543, 5,580,732, 5,624,800, 5,807,682, 6,759,202, 6,756,204, 6,734,296, 6,395,486, U.S. patent application Ser. Nos. 09/584,905 and 09/724,755, and Published U.S. Patent Application No. US 2003-0190646 A1. Exemplary kits in which amplification products may be used include, but are not limited to, TaqMan® SNP Genotyping Products, Applied Biosystems Part number 4331183; TaqMan® Pre-Designed SNP Genotyping Assays, Applied Biosystems Part number 4351370; TaqMan® Gene Expression Assays, Applied Biosystems Part number 4331182; ABI PRISM® SNaPshot™ Multiplex Kit (for sequencing), Applied Biosystems Part number 4323151; AmpFLSTR® Identifiler® PCR Amplification Kit, Applied Biosystems Part number 4322288.

In certain embodiments, amplification products are treated before they are used in a downstream process. For example, and not limitation, in certain embodiments, amplification products are heated prior to use in a downstream process.

### EXAMPLE 1

An Exemplary Amplification Reaction

Solution A was prepared by adding 5 μl of 500 μM primer DR10D (5′-SSNNNNNNNN-3′), 2 μl of neat DMSO (American Type Culture Collection (ATCC), Rockville, Md.), 0.5 μl of 100 mM dNTPs (25 mM each: dATP, dCTP, dGTP and dTTP) (Applied Biosystems, Foster City, Calif.), 4 μl of 10× Thermopol buffer (New England Biolabs, Beverly, Mass.) and 1 μl of a 10 ng/μl stock of Human genomic DNA template (Clontech, Palo Alto, Calif.) to a final solution volume of 40 μl.

Solution B was prepared by adding 2.2 μl of Bst DNA Polymerase Large-Fragment stock enzyme (New England Biolabs, Beverly, Mass. (NEB catalog #M0275L, 8,000 Units/ml)) and 1.0 μl of 10× Thermopol buffer (New England Biolabs, Beverly, Mass.) to a final volume of 10 μl.

Solution A was added into each well of a 96-well thermocycler plate. The plate wells were sealed, and the plate was placed into an ABI 9700 thermocycler (Applied Biosystems, Foster City, Calif.). The plate was heated at 94° C. for 3 minutes. Following the heating step, the plate was cooled to 4° C. for 1 to 5 minutes. The plate was then heated to 50° C. and the wells were unsealed. Once the wells were unsealed, 10 μl of a solution B was added with gentle mixing. The wells were resealed, and the plate was maintained at 50° C. for 5 hours.

The reaction composition in each well comprised the following:

1× ThermoPol (20 mM Tris-HCl pH 8.8, 10 mM KCl, 10 mM (NH₄)SO₄, 2 mM MgSO₄, 0.1% Triton X-100)

4% neat DMSO

50 μM Primer DR10D

1 mM dNTPs (0.25 mM each: dATP, dCTP, dGTP and dTTP)

0.35 U/μl Bst DNA Polymerase (Large-Fragment)

10 ng Human genomic DNA template (Clontech, Palo Alto, Calif.)

Following amplification, amplification products were analyzed according to examples 2 to 4.

### EXAMPLE 2

Agarose Gel Electrophoresis of Amplification Products

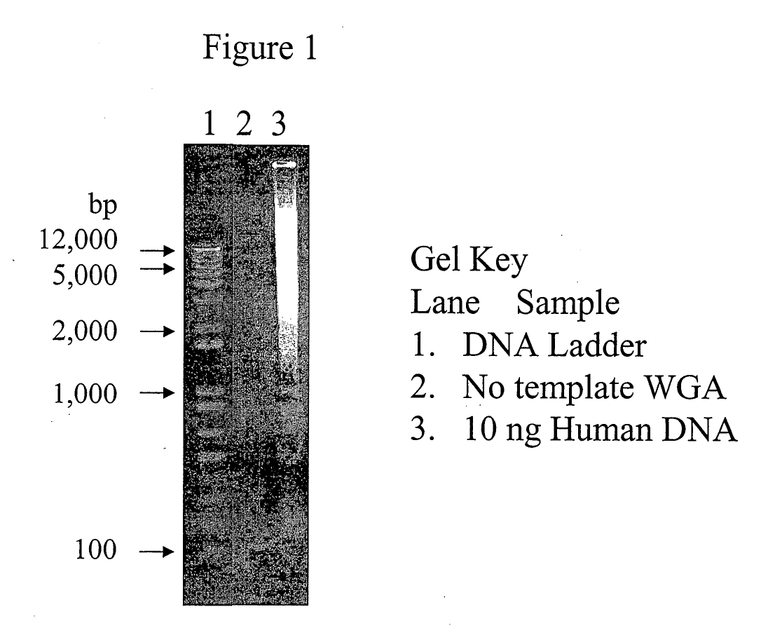

The results described in this Example are illustrated in FIG. 1.

An amplification reaction comprising 10 ng of human genomic DNA template was conducted as described in Example 1. A second amplification reaction was conducted as described in Example 1 except that the second amplification reaction had no template nucleic acids. That second amplification reaction served as a “No Template Control.”

Amplification products were heated to 93° to 95° C. for 10 minutes. After cooling to ambient temperature, a 1 μl aliquot of amplification product was mixed with 1.5 μl water and 2.5 μl of 2× gel loading buffer. Samples and a molecular weight DNA ladder (1 KB Plus Ladder (Invitrogen, Carlsbad, Calif.)) were loaded into wells on a 1.0% agarose gel (1×TBE buffer). The samples were electrophoresed at 10V/cm for 30 minutes. The gel was stained with a 0.5 μg/ml ethidium bromide solution for 15 minutes and photographed over a UV transilluminator. FIG. 1 displays from left to right the molecular weight ladder, the no template products, and the human genomic DNA products. The no template control reaction produced almost no amplified DNA that could be detected on the agarose gel. The Amplification reaction containing human genomic DNA template produced a distribution of DNA products corresponding to DNA Ladder markers from 400 bp to an excess of 12,000 bp.

### EXAMPLE 3

Picogreen Analysis of Amplification Products

An amplification reaction comprising 10 ng of human genomic DNA template was conducted as described in Example 1. A second amplification reaction was conducted as described in Example 1 except that the second amplification reaction had no template nucleic acids. That second amplification reaction served as a No Template Control.

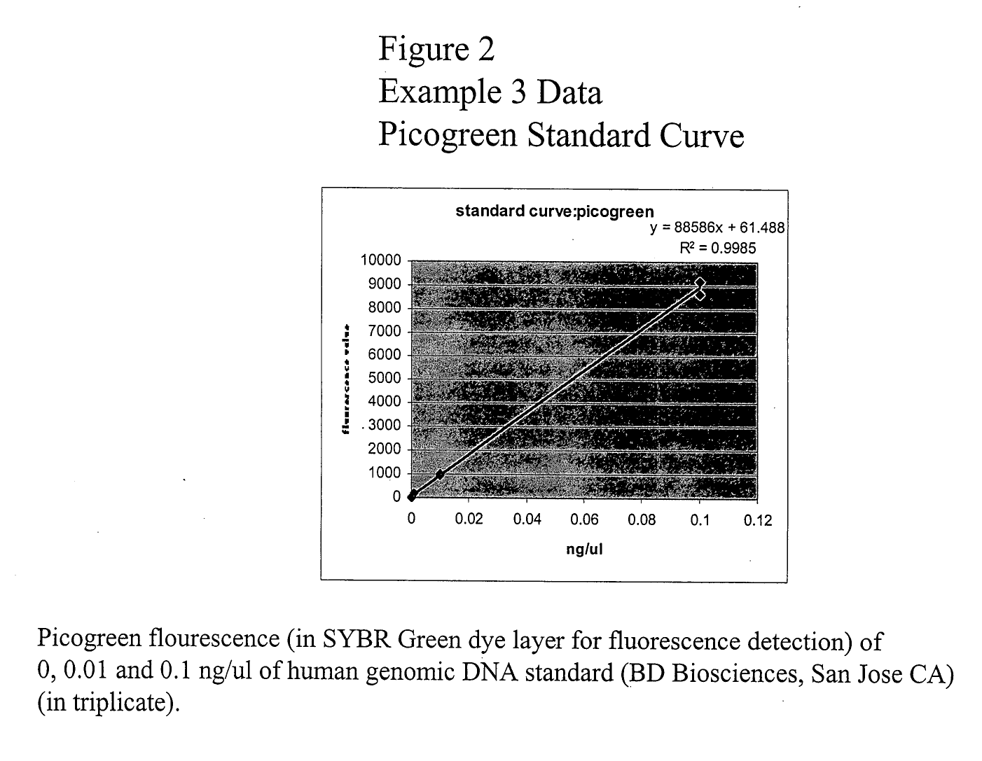

Following amplification, reaction products were analyzed using a picogreen assay (Molecular Probes, Picogreen® dsDNA quantitation kit catalog #P7589) to determine the yield of amplified DNA. The reaction products from the amplification were diluted 1:50 in TE, followed by a second dilution of 1:50 in TE (overall dilution was 1:2500). The picogreen stock was diluted 1:200 in TE. 50 μl of the 1:2500 dilution was added to 50 μl of the diluted Picogreen mix in 96-well PCR plates to create a 100 μl Picogreen reaction mix. The Picogreen reaction mix was incubated at room temperate for 1 minute. Following incubation, the products of the Picogreen reaction mix were analyzed on an ABI 7700 sequence detection system using the SYBR dye layer for fluorescence detection (Applied Biosystems, Foster City, Calif.). Otherwise, the picogreen analysis was conducted according to the instructions in the Picogreen® dsDNA quantitation kit. A standard curve was constructed with known amounts of control DNA as part of the picogreen analysis. All samples were measured in triplicate. The different data points on the standard curve were at 0, 0.1, 1, 10, and 10 ng per 100 μl, with 0 ng/100 μl being the No Template Control. The standard curve was used to convert fluorescence measurements for experimental samples to ng/μl concentrations and total yields of amplified DNA, as shown in FIG. 2.

### EXAMPLE 4

TaqMan Analysis of Amplification Products

An amplification reaction comprising 10 ng of human genomic DNA template was conducted as described in Example 1. A second amplification reaction was conducted as described in Example 1 except that the second amplification reaction had no template nucleic acids. That second amplification reaction served as a No Template Control.

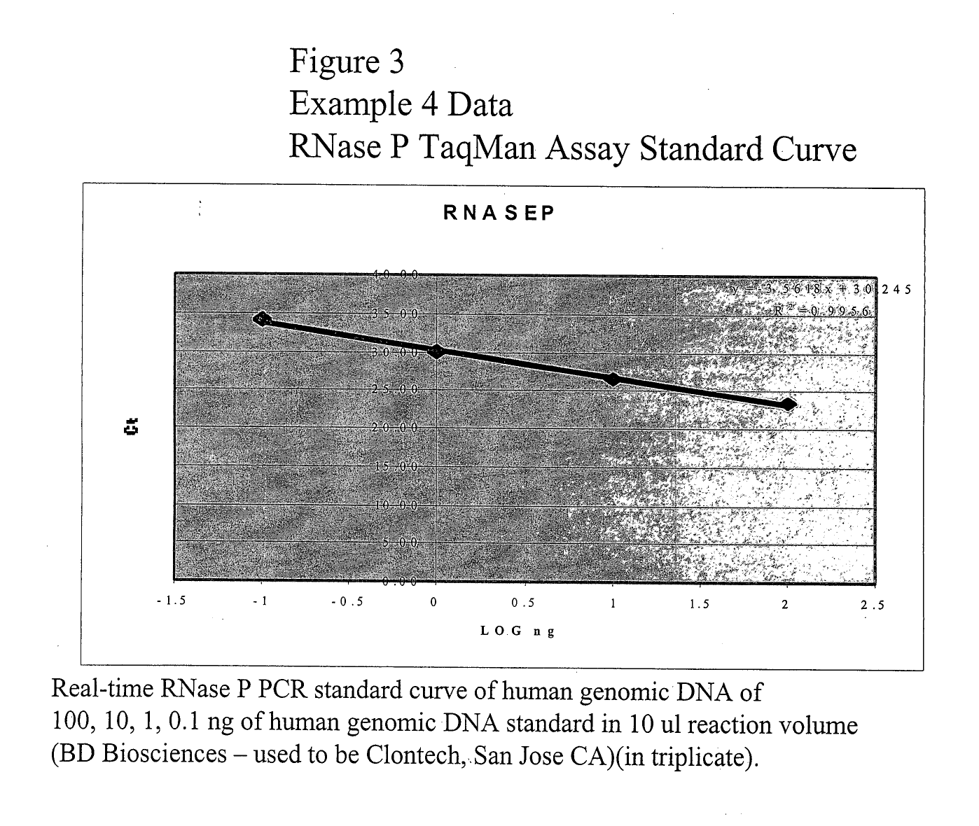

Following amplification, reaction products were analyzed using a TaqMan assay for RNASE P (Applied Biosystems, Foster City, Calif., catalog #4316844). The reaction products were heated at 95° C. for 1.0 minutes. Following heating, the reaction products were diluted 1:50 in TE. 2.5 μl of the diluted reaction products were used per 10 μl TaqMan assay. Otherwise, the TaqMan assay was conducted according the manufacturer's instructions. Each TaqMan assay measures the amount of DNA at a particular position, in this case, the RNASE P gene. A series of known amounts of unamplified genomic DNA were also assayed in order to generate a standard curve for determining yield and fold amplification. The known amounts of DNA used were 0, 0.1, 1, 10 and 100 ng per 10 μl TaqMan assay. The standard curve was used to convert CT measurements for experimental samples to ng/ul concentrations and determining fold amplification relative to a starting input of 10 ng. That standard curve is shown in FIG. 3.

### EXAMPLE 5

Amplification Analysis Using a First Series of Primer Sets

Fifteen different primer sets were individually tested for their ability to amplify nucleic acids in amplification reactions. One primer set was used for each different amplification reaction. The fifteen primer sets that were used appear in Table 1 below.

TABLE 1

| PRIMER SET | SEQUENCE |
|---|---|
| DR01 | 55NNNNNN |
| DR02 | 55NNNNNNNN |
| DR03 | 55NNNNNNtN |
| DR04 | 55NNNNNNNNtN |
| DR05 | IINNNN |
| DR06 | IINNNNNN |
| DR07 | IINNNNNNNN |
| DR08 | IINNNNNNNNNN |
| DR09 | IINNNNNtN |
| DR10 | NNNNNNNtN |
| DR11 | NNNNNNNNNtN |
| DR12 | NNNNNNNNNNNtN |
| DR13 | NNNNNNNNNNNNNNtN |
| DR14 | NNNNNNNNNNNNNNN |
| DR15 | NNNNNNNNNNNNNNNNNN |

Separate solutions of solution A were prepared by adding 5 μl of a 500 μM stock of one of primer sets DR1 to DR15, 2 μl of neat DMSO (American Type Culture Collection (ATCC), Rockville, Md.), 0.5 μl of 100 mM dNTPs (25 mM each: dATP, dCTP, dGTP and dTTP) (Applied Biosystems, Foster City, Calif.), 4 μl of 10× Thermopol buffer (New England Biolabs, Beverly, Mass.), and an amount of human genomic DNA as set forth below, to a final solution volume of 40 μl. For primer sets DR1, DR2, and DR4 to DR15, one solution A was formed comprising 0 ng of Human genomic DNA template (Clontech, Palo Alto, Calif.); one solution A was formed comprising 0.1 ng of Human genomic DNA template (Clontech, Palo Alto, Calif.); and one solution A was formed comprising 10 ng of Human genomic DNA template (Clontech, Palo Alto, Calif.). For primer set DR3, one solution A was formed comprising 0 ng of Human genomic DNA template (Clontech, Palo Alto, Calif.); one solution A was formed comprising 0.1 ng of Human genomic DNA template (Clontech, Palo Alto, Calif.); one solution A was formed comprising 1 ng of Human genomic DNA template (Clontech, Palo Alto, Calif.); one solution A was formed comprising 10 ng of Human genomic DNA template (Clontech, Palo Alto, Calif.); and one solution A was formed comprising 100 ng of Human genomic DNA template (Clontech, Palo Alto, Calif.).

Solution B was prepared by adding 2.2 μl of Bst DNA Polymerase Large-Fragment stock enzyme (New England Biolabs, Beverly, Mass. (NEB catalog #M0275L, 8,000 Units/ml)) and 1.0 μl of 10× Thermopol buffer (New England Biolabs, Beverly, Mass.) to a final volume of 10 μl.

Solution A was added into a well of a 96-well thermocycler plate, such that there were 47 wells comprising a solution A in the 96 well plate, each well comprising a different primer set. The plate wells were sealed, and the plate was placed into an ABI 9700 thermocycler (Applied Biosystems, Foster City, Calif.). The plate was heated at 94° C. for 3 minutes. Following the heating step, the plate was cooled to 4° C. for 1 to 5 minutes. The plate was then heated to 50° C. and the wells were unsealed. Once the wells were unsealed, 10 μl of solution B was added with gentle mixing. The wells were resealed, and the plate was maintained at 50° C. for 5 hours.

The reaction composition in each well comprised the following:

1× ThermoPol (20 mM Tris-HCl pH 8.8, 10 mM KCl, 10 mM (NH₄)SO₄, 2 mM MgSO₄, 0.1% Triton X-100)

4% neat DMSO

50 μM Primer

1 mM dNTPs (0.25 mM each: dATP, dCTP, dGTP and dTTP)

0.35 U/μl Bst DNA Polymerase (Large-Fragment)

Human genomic DNA template (0 ng, 0.1 ng, 1 ng, 10 ng, or 100 ng) (Clontech, Palo Alto, Calif.)

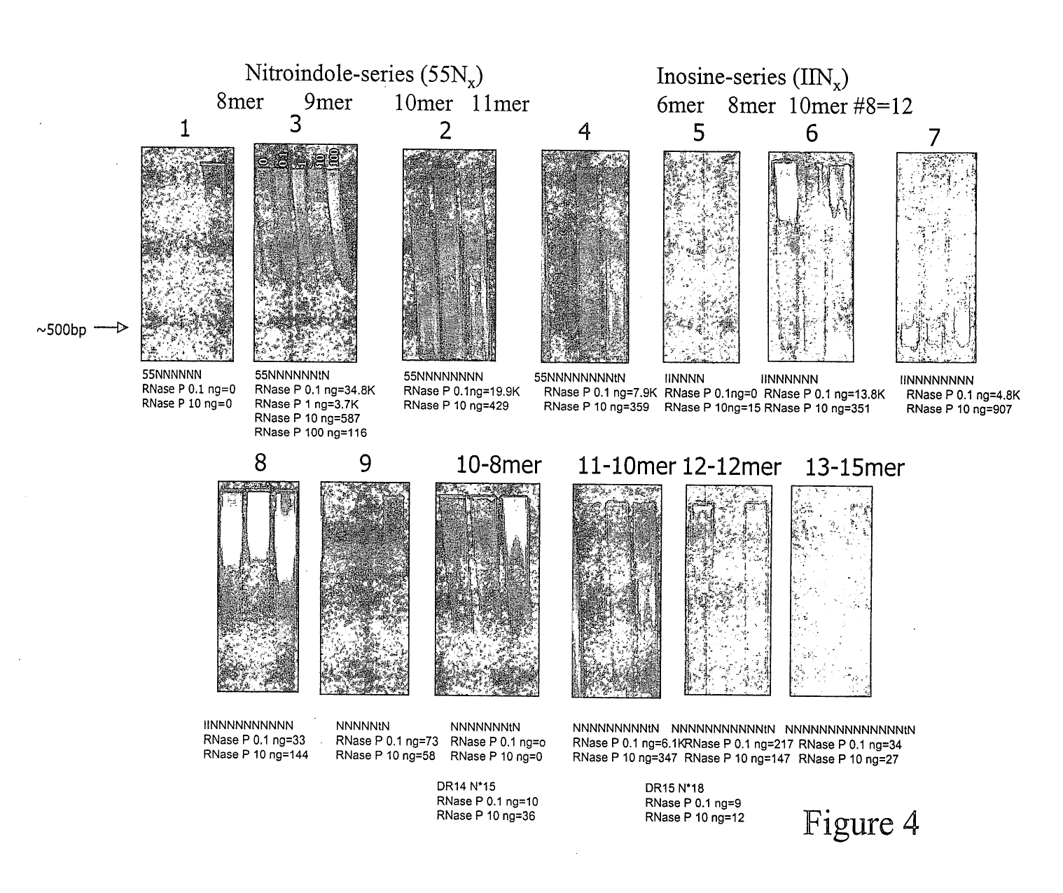

Following amplification, the products of each amplification reaction were analyzed for yield and fold amplification using the RNASE P TaqMan probe as described in Example 4. Also, amplification products from reaction compositions containing primer sets DR1 to DR13 were subjected to agarose gel electrophoresis as described in Example 2. Those results are shown in FIG. 4.

### EXAMPLE 6

Effect of Hot Start on Amplification Reactions

The effect of heating the reactants before adding the polymerase (“a hot start”) versus not heating the reactants before adding the polymerase (“a cold start”) was compared for primer sets DR2, DR3, DR4, DR6, DR7, and DR10. Amplification reactions were performed as follows:

Separate solutions of solution A were prepared by adding 5 μl of a 500 μM stock of one of primer sets DR2, DR3, DR4, DR6, DR7, or DR10, 2 μl of neat DMSO (American Type Culture Collection (ATCC), Rockville, Md.), 0.5 μl of 100 mM dNTPs (25 mM each: dATP, dCTP, dGTP and dTTP) (Applied Biosystems, Foster City, Calif.), 4 μl of 10× Thermopol buffer (New England Biolabs, Beverly, Mass.), and an amount of human genomic DNA as set forth below, to a final solution volume of 40 μl. For each primer set, two solutions of solution A comprised 0 ng of nucleic acid template; two solutions of solution A comprised 0.1 ng of nucleic acid template; and two solutions of solution A comprised 10 ng of nucleic acid template.

Solution B was prepared by adding 2.2 μl of Bst DNA Polymerase Large-Fragment stock enzyme (New England Biolabs, Beverly, Mass. (NEB catalog #M0275L, 8,000 Units/ml)) and 1.0 μl of 10× Thermopol buffer (New England Biolabs, Beverly, Mass.) to a final volume of 10 μl.

Solution A was added into a well of a 96-well thermocycler plate, such that there were 36 wells comprising a solution A in the 96 well plate, each well comprising a different primer set. For each primer set, one solution A comprising 0 ng of nucleic acid template, one solution A comprising 0.1 ng of nucleic acid template, and one solution A comprising 10 ng of nucleic acid template, were subjected to a “hot start” reaction in which the solution A was heated to 50° C. for two minutes before solution B was added. Also, for each primer set, one solution A comprising 0 ng of nucleic acid template, one solution A comprising 0.1 ng of nucleic acid template, and one solution A comprising 10 ng of nucleic acid template, were subjected to a “cold start” reaction in which the solutions A and B were combined at room temperature.

For both the “hot start” reactions and the “cold start” reactions, 10 μl of solution B was added with gentle mixing to the well containing solution A. The wells were sealed, and the plate was maintained at 50° C. for 5 hours.

The reaction composition in each well comprised the following:

1× ThermoPol (20 mM Tris-HCl pH 8.8, 10 mM KCl, 10 mM (NH₄)SO₄, 2 mM MgSO₄, 0.1% Triton X-100)

4% neat DMSO

50 μM Primer

1 mM dNTPs (0.25 mM each: dATP, dCTP, dGTP and dTTP)

0.35 U/μl Bst DNA Polymerase (Large-Fragment)

Human genomic DNA template (0 ng, 0.1 ng, or 10 ng) (Clontech, Palo Alto, Calif.)

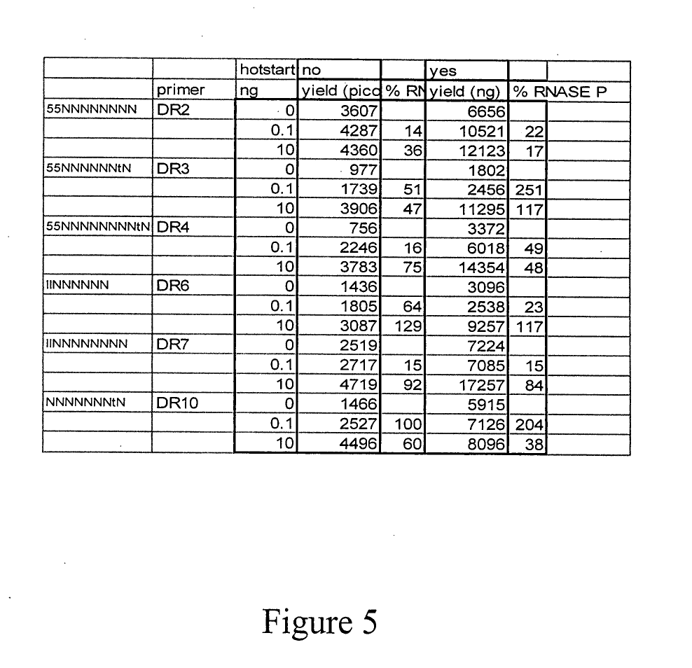

After amplification, reaction products from each amplification reaction were analyzed using Picogreen assays and TaqMan assays. The Picogreen assays were performed as described in Example 3. The TaqMan assays were performed as described in Example 4, and used an RNAse P probe as described in that example. FIG. 5 shows those results.

### EXAMPLE 7

Effect of Heating on Reaction Products

The effect of heating the reaction products after amplification was also analyzed. Fifteen different primer sets were tested. The tested primer sets were DR1, DR2, DR3, DR4, DR5, DR6, DR7, DR8, DR9, DR10, DR11, DR12, DR13, DR14, and DR15. Reaction compositions were formed for each primer set as follows:

Separate solutions of solution A were prepared by adding 5 μl of a 500 μM stock of one of primer sets DR1 to DR15, 2 μl of neat DMSO (American Type Culture Collection (ATCC), Rockville, Md.), 0.5 μl of 100 mM dNTPs (25 mM each: dATP, dCTP, dGTP and dTTP) (Applied Biosystems, Foster City, Calif.), 4 μl of 10× Thermopol buffer (New England Biolabs, Beverly, Mass.), and an amount of human genomic DNA as set forth below, to a final solution volume of 40 μl. For each primer set, one solution A was formed comprising 0 ng of Human genomic DNA template (Clontech, Palo Alto, Calif.); one solution A was formed comprising 0.1 ng of Human genomic DNA template (Clontech, Palo Alto, Calif.); and one solution A was formed comprising 10 ng of Human genomic DNA template (Clontech, Palo Alto, Calif.).

Solution B was prepared by adding 2.2 μl of Bst DNA Polymerase Large-Fragment stock enzyme (New England Biolabs, Beverly, Mass. (NEB catalog #M0275L, 8,000 Units/ml)) and 1.0 μl of 10× Thermopol buffer (New England Biolabs, Beverly, Mass.) to a final volume of 10 μl.

Solution A was added into a well of a 96-well thermocycler plate, such that there were 45 wells comprising a solution A in the 96 well plate, each well comprising a different primer set. The plate wells were sealed, and the plate was placed into an ABI 9700 thermocycler (Applied Biosystems, Foster City, Calif.). The plate was heated at 94° C. for 3 minutes. Following the heating step, the plate was cooled to 4° C. for 1 to 5 minutes. The plate was then heated to 50° C. and the wells were unsealed. Once the wells were unsealed, 10 μl of solution B was added with gentle mixing. The wells were resealed, and the plate was maintained at 50° C. for 5 hours.

The reaction composition in each well comprised the following:

1× ThermoPol (20 mM Tris-HCl pH 8.8, 10 mM KCl, 10 mM (NH₄)SO₄, 2 mM MgSO₄, 0.1% Triton X-100)

4% neat DMSO

50 μM Primer

1 mM dNTPs (0.25 mM each: dATP, dCTP, dGTP and dTTP)

0.35 U/μl Bst DNA Polymerase (Large-Fragment)

Human genomic DNA template (0 ng, 0.1 ng, or 10 ng) (Clontech, Palo Alto, Calif.)

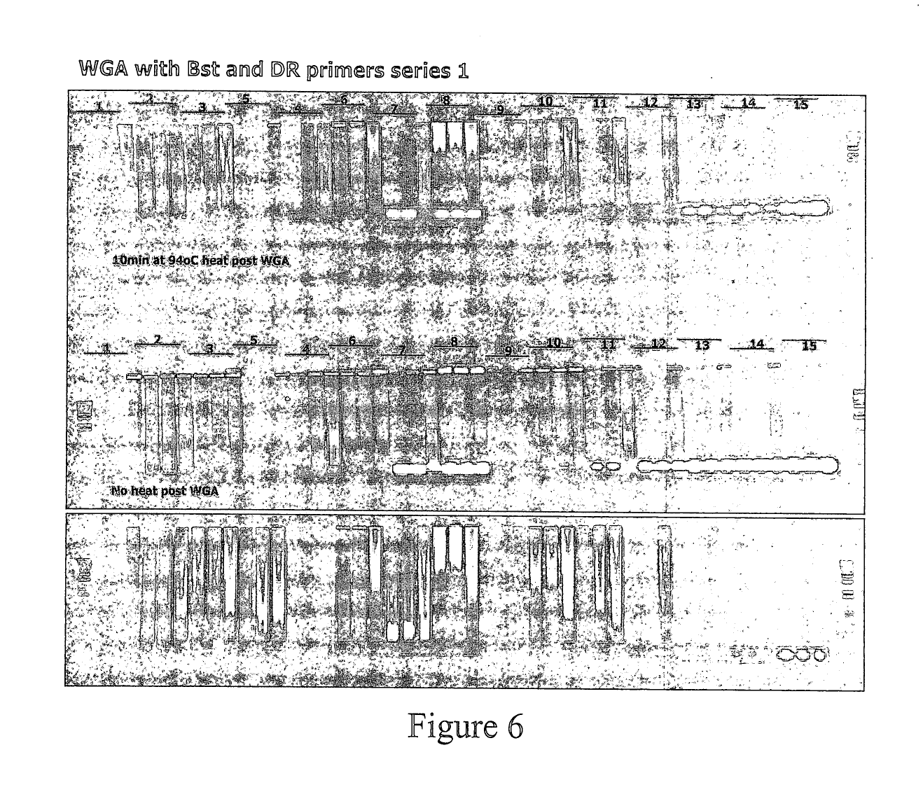

After performing the amplification reactions, 2.5 μl of reaction products were removed from each reaction composition and heated for 10 minutes at 94° C. The heated reaction products were then loaded onto an agarose gel and electrophoresed. The heated reaction products were compared to reaction products of the same amplification reaction that were not heated for 10 minutes after the amplification process. Those results are shown in FIG. 6.

### EXAMPLE 8

Amplification Analysis Using a Second Series of Primer Sets

Thirty six different primer sets were individually tested for their ability to amplify nucleic acids in an amplification reaction. One primer set was used for each different amplification reaction. The thirty six primer sets that were used appear in Table 2 below.

TABLE 2

| PRIMER SET | SEQUENCE |
|---|---|
| DR01B | CTNNNNNN |
| DR02B | CTNNNNNNN |
| DR03B | CTNNNNNNNN |
| DR04B | CTCNNNNN |
| DR05B | CTCNNNNNN |
| DR06B | CTCNNNNNNN |
| DR07B | NNNNNNTC |
| DR08B | NNNNNNNTC |
| DR09B | NNNNNNNNTC |
| DR10B | NNNNNCTC |
| DR11B | NNNNNNCTC |
| DR12B | NNNNNNNCTC |
| DR13B | GANNNNNN |
| DR14B | GANNNNNNN |
| DR15B | GANNNNNNNN |
| DR16B | GAGNNNNN |
| DR17B | GAGNNNNNN |
| DR18B | GAGNNNNNNN |
| DR19B | NNNNNNAG |
| DR20B | NNNNNNNAG |
| DR21B | NNNNNNNNAG |
| DR22B | NNNNNGAG |
| DR23B | NNNNNNGAG |
| DR24B | NNNNNNNGAG |
| DR25B | NNGCCGNN |
| DR26B | NNNGCCGNNN |
| DR27B | NNGAAGNN |
| DR28B | NNNGAAGNNN |
| DR29B | NNGAGANN |
| DR30B | NNNGAGANNN |
| DR31B | NNTCCTNN |
| DR32B | NNNTCCTNNN |
| DR33B | NNTTTTNN |
| DR34B | NNNTTTTNNN |
| DR35B | NNCCCCNN |
| DR36B | NNNCCCCNNN |

Amplification reactions were performed as follows:

Separate solutions of solution A were prepared by adding 5 μl of a 500 μM stock of one of primer sets DR1B to DR36B, 2 μl of neat DMSO (American Type Culture Collection (ATCC), Rockville, Md.), 0.5 μl of 100 mM dNTPs (25 mM each: dATP, dCTP, dGTP and dTTP) (Applied Biosystems, Foster City, Calif.), 4 μl of 10× Thermopol buffer (New England Biolabs, Beverly, Mass.), and an amount of human genomic DNA as set forth below, to a final solution volume of 40 μl. For each primer set, one solution A was formed comprising 0 ng of Human genomic DNA template (Clontech, Palo Alto, Calif.); and one solution A was formed comprising 10 ng of Human genomic DNA template (Clontech, Palo Alto, Calif.).

Solution B was prepared by adding 2.2 μl of Bst DNA Polymerase Large-Fragment stock enzyme (New England Biolabs, Beverly, Mass. (NEB catalog #M0275L, 8,000 Units/ml)) and 1.0 μl of 10× Thermopol buffer (New England Biolabs, Beverly, Mass.) to a final volume of 10 μl.

Solution A was added into a well of a 96-well thermocycler plate, such that there were 72 wells comprising a solution A in the 96 well plate, each well comprising a different primer set. The plate wells were sealed, and the plate was placed into an ABI 9700 thermocycler (Applied Biosystems, Foster City, Calif.). The plate was heated at 94° C. for 3 minutes. Following the heating step, the plate was cooled to 4° C. for 1 to 5 minutes. The plate was then heated to 50° C. and the wells were unsealed. Once the wells were unsealed, 10 μl of solution B was added with gentle mixing. The wells were resealed, and the plate was maintained at 50° C. for 5 hours.

The reaction composition in each well comprised the following:

1× ThermoPol (20 mM Tris-HCl pH 8.8, 10 mM KCl, 10 mM (NH₄)SO₄, 2 mM MgSO₄, 0.1% Triton X-100)

4% neat DMSO

50 μM Primer

1 mM dNTPs (0.25 mM each: dATP, dCTP, dGTP and dTTP)

0.35 U/μl Bst DNA Polymerase (Large-Fragment)

Human genomic DNA template (0 ng or 10 ng) (Clontech, Palo Alto, Calif.)

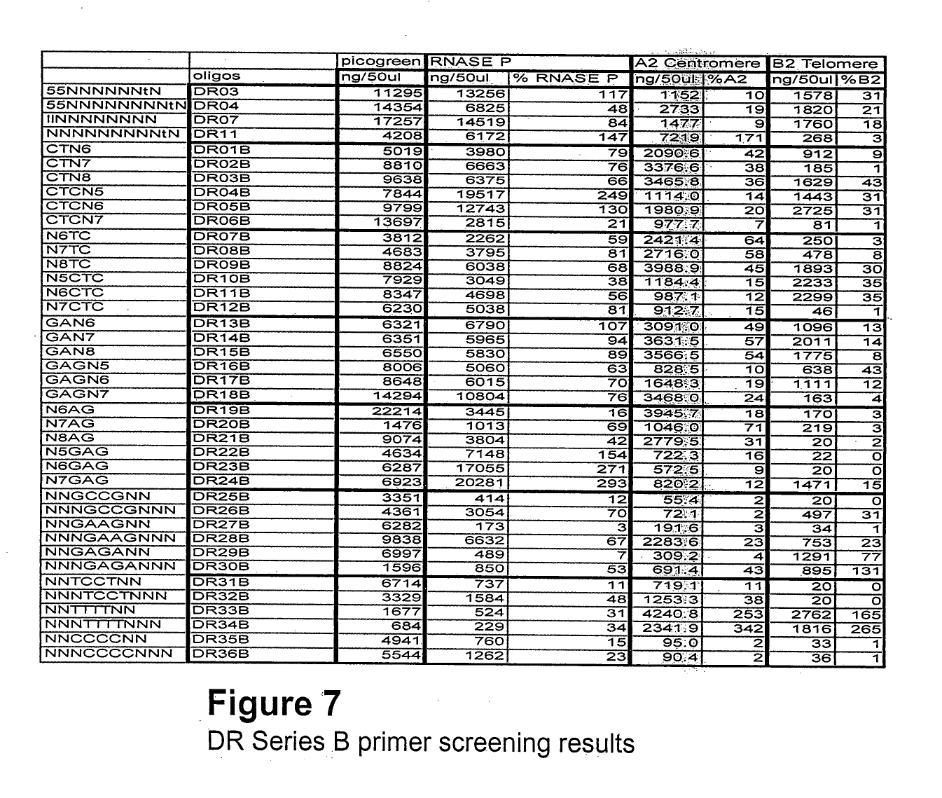

Amplification products from each amplification reaction were analyzed using a picogreen assay and three different TaqMan assays. Picogreen assays were carried out as described in Example 3. TaqMan assays were performed as described in Example 4. TaqMan assays were performed with three different TaqMan probes. Each different TaqMan probe was specific for a different chromosomal position. One TaqMan probe was specific for the RNaseP locus located in a euchromatic region of chromosome 6. The probe for the RNase P locus is described in Example 4. The second TaqMan probe was specific for a centromere site on chromosome 6. The probe for the centromere site corresponds to assay hCV7814872:Chromosome 6, Celera position 57,598,458; public position 55,933,666 Collagenase type XXI alpha I, 6p11.1. And the third TaqMan probe was specific for a telomere site on chromosome 1. The probe for the telomere site corresponds to assay hCV349932 Chromosome 1, Celera position 221,862,772, public position 243,790,691 (1q44). (The hCV numbers correspond to Celera SNP Ids for assays available from Applied Biosystems.) The results of those assays are shown in FIG. 7.

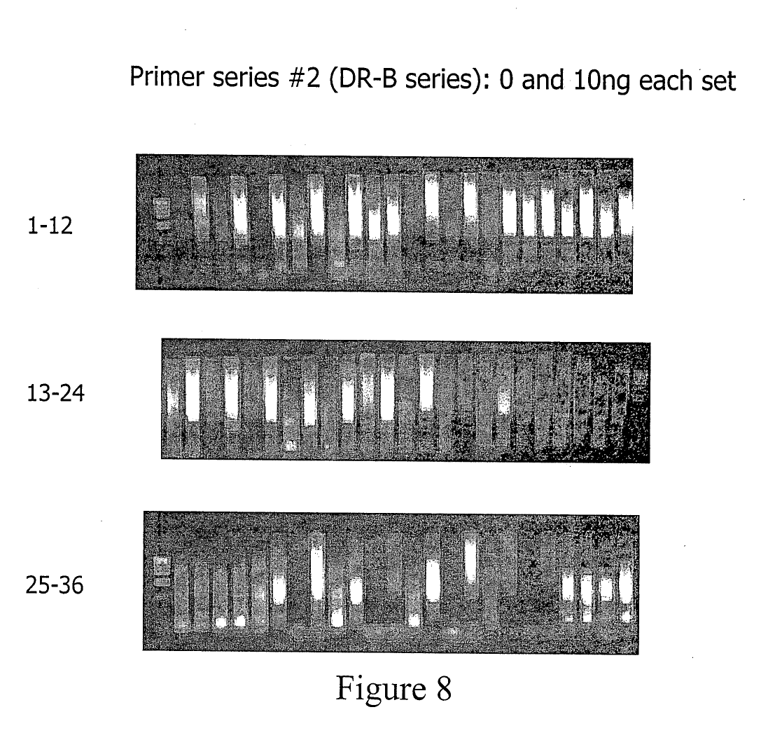

Amplification products were also analyzed by agarose gel electrophoresis. Agarose gel electrophoresis was performed as described in Example 2. A 0.5 μl aliquot of each reaction product of the 36 different amplification reactions was removed and electrophoresed on an agarose gel to evaluate the amplification efficiency and the distribution in size of the amplification products as shown in FIG. 8. The first lanes in the top and bottom panels contain 1 Kb Plus DNA ladder. All other lanes alternate between 0 and 10 ng of input genomic DNA for the reaction composition. Primer sets for each panel are shown to the left.

### EXAMPLE 9

Amplification Time Course

Primer sets DR03, DR11, DR03B, DR07B, or DR15B were each evaluated in separate amplification reactions at five different time points. Amplification reactions were performed as follows:

Separate solutions of solution A were prepared by adding 5 μl of a 500 μM stock of one of primer sets DR03, DR11, DR03B, DR07B, and DR15B, 2 μl of neat DMSO (American Type Culture Collection (ATCC), Rockville, Md.), 0.5 μl of 100 mM dNTPs (25 mM each: dATP, dCTP, dGTP and dTTP) (Applied Biosystems, Foster City, Calif.), 4 μl of 10× Thermopol buffer (New England Biolabs, Beverly, Mass.), and 10 ng of Human genomic DNA template (Clontech, Palo Alto, Calif.) to a final solution volume of 40 μl.

Solution B was prepared by adding 2.2 μl of Bst DNA Polymerase Large-Fragment stock enzyme (New England Biolabs, Beverly, Mass. (NEB catalog #M0275L, 8,000 Units/ml)) and 1.0 μl of 10× Thermopol buffer (New England Biolabs, Beverly, Mass.) to a final volume of 10 μl.

Solution A was added into a well of a 96-well thermocycler plate, such that there were 5 wells comprising a solution A in the 96 well plate, each well comprising a different primer set. The plate wells were sealed, and the plate was placed into an ABI 9700 thermocycler (Applied Biosystems, Foster City, Calif.). The plate was heated at 94° C. for 3 minutes. Following the heating step, the plate was cooled to 4° C. for 1 to 5 minutes. The plate was then heated to 50° C. and the wells were unsealed. Once the wells were unsealed, 10 μl of solution B was added with gentle mixing.

Five separate reaction compositions were prepared for each primer set and incubated at 50° C. for either 0, 2, 5, 8 or 22 hours before storing at 4° C.

The reaction composition in each well comprised the following:

1× ThermoPol (20 mM Tris-HCl pH 8.8, 10 mM KCl, 10 mM (NH₄)SO₄, 2 mM MgSO₄, 0.1% Triton X-100)

4% neat DMSO 50 μM Primer

1 mM dNTPs (0.25 mM each: dATP, dCTP, dGTP and dTTP)

0.35 U/μl Bst DNA Polymerase (Large-Fragment)

Human genomic DNA template (10 ng) (Clontech, Palo Alto, Calif.)

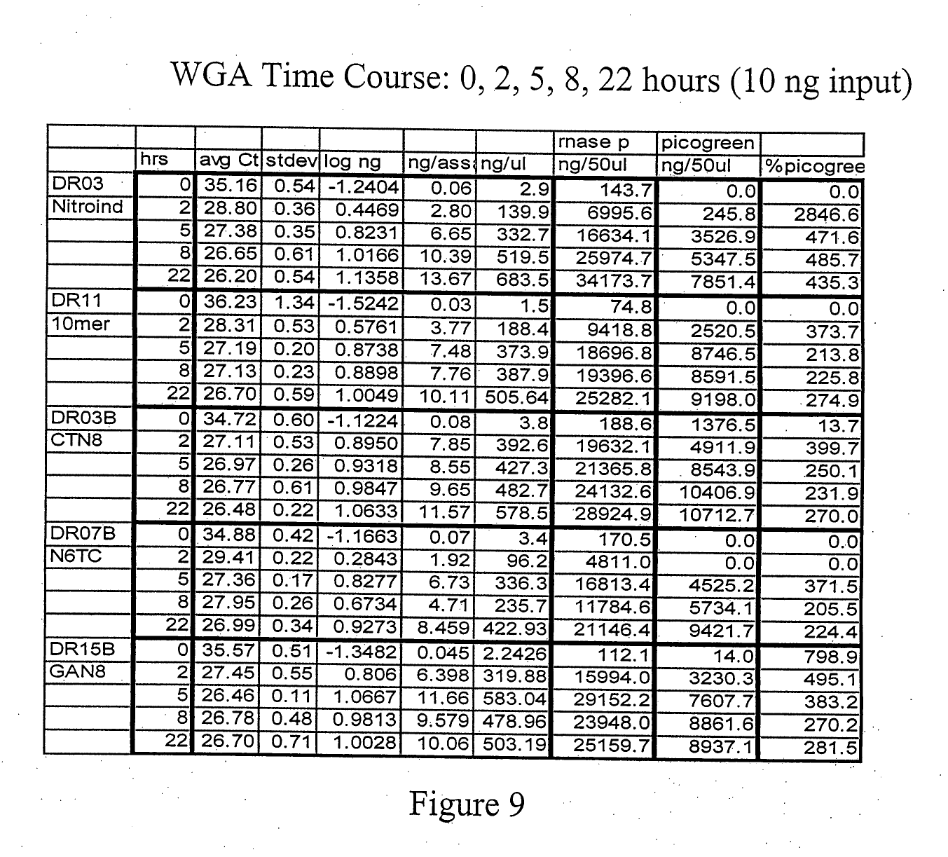

The samples were analyzed using Picogreen assays and TaqMan assays. The Picogreen assays were performed as described in Example 3. The TaqMan assays were performed as described in Example 4, and used an RNAse P probe as described in that example. The time course is shown in FIG. 9.

### EXAMPLE 10

Chromosomal Amplification Bias Assay

The amplification profiles of primers DR03, DR11, DR03B, DR04B, DR07B, DR15B, and DR24B were evaluated using a chromosomal bias assay. An amplification reaction was performed for each primer set as follows.

Separate solutions of solution A were prepared by adding 5 μl of a 500 μM stock of one of primer sets DR03, DR11, DR03B, DR04B, DR07B, DR15B, and DR24B, 2 μl of neat DMSO (American Type Culture Collection (ATCC), Rockville, Md.), 0.5 μl of 100 mM dNTPs (25 mM each: dATP, dCTP, dGTP and dTTP) (Applied Biosystems, Foster City, Calif.), 4 μl of 10× Thermopol buffer (New England Biolabs, Beverly, Mass.), and 10 ng of Human genomic DNA template (Clontech, Palo Alto, Calif.) to a final solution volume of 40 μl.

Solution B was prepared by adding 2.2 μl of Bst DNA Polymerase Large-Fragment stock enzyme (New England Biolabs, Beverly, Mass. (NEB catalog #M0275L, 8,000 Units/ml)) and 1.0 μl of 10× Thermopol buffer (New England Biolabs, Beverly, Mass.) to a final volume of 10 μl.

Solution A was added into a well of a 96-well thermocycler plate, such that there were 7 wells comprising a solution A in the 96 well plate, each well comprising a different primer set. The plate wells were sealed, and the plate was placed into an ABI 9700 thermocycler (Applied Biosystems, Foster City, Calif.). The plate was heated at 94° C. for 3 minutes. Following the heating step, the plate was cooled to 4° C. for 1 to 5 minutes. The plate was then heated to 50° C. and the wells were unsealed. Once the wells were unsealed, 10 μl of solution B was added with gentle mixing. The wells were resealed, and the plate was maintained at 50° C. for 5 hours.

The reaction composition in each well comprised the following:

1× ThermoPol (20 mM Tris-HCl pH 8.8, 10 mM KCl, 10 mM (NH₄)SO₄, 2 mM MgSO₄, 0.1% Triton X-100)

4% neat DMSO

50 μM Primer

1 mM dNTPs (0.25 mM each: dATP, dCTP, dGTP and dTTP)

0.35 U/μl Bst DNA Polymerase (Large-Fragment)

Human genomic DNA template (10 ng) (Clontech, Palo Alto, Calif.)

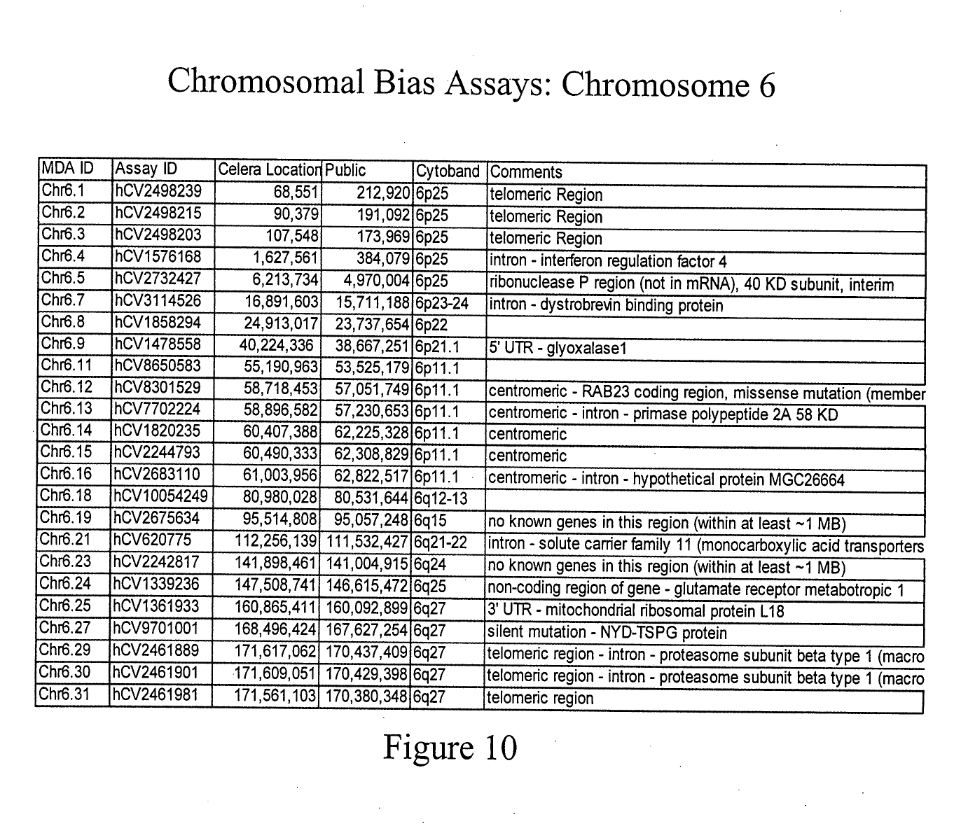

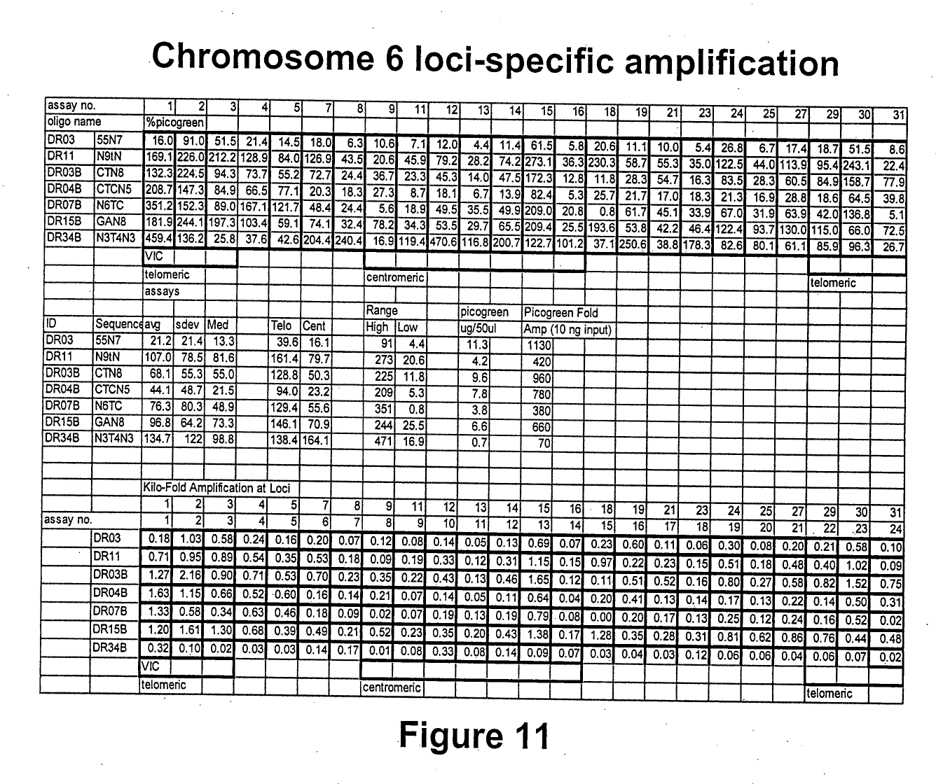

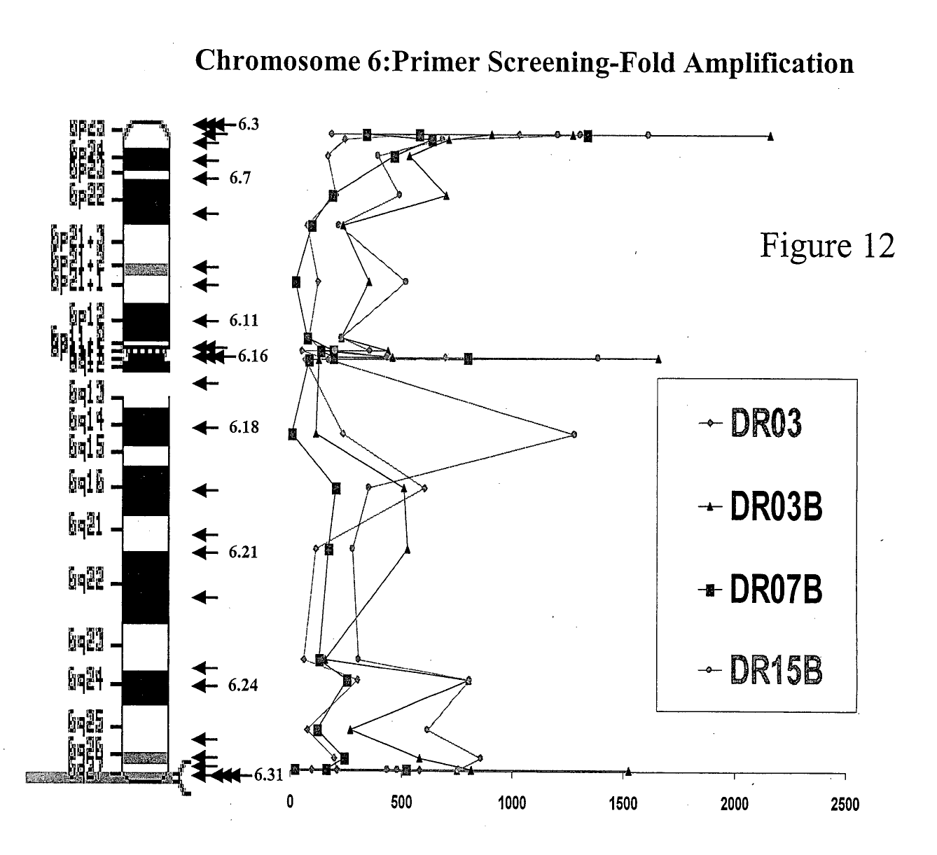

Following amplification, amplification products from each of those amplification reactions were then used in 24 separate TaqMan assays. Each TaqMan assay was performed as described in example 4. The following TaqMan probes were used in the assays: Chr6.1 (hCV2498239), Chr6.2 (hCV2498215), Chr6.3 (hCV2498203), Chr6.4 (hCV1576168), Chr6.5 (hCV2732427), Chr6.7 (hCV3114526), Chr6.8 (hCV1858294), Chr6.9 (hCV1478558), Chr6.11 (hCV8650583), Chr6.12 (hCV8301529), Chr6.13 (hCV7702224), Chr6.14 (hCV1820235), Chr6.15 (hCV2244793), Chr6.16 (hCV2683110), Chr6.18 (hCV10054249), Chr6.19 (hCV2675634), Chr6.21 (hCV620775), Chr6.23 (hCV2242817), Chr6.24 (hCV1339236), Chr6.25 (hCV1361933), Chr6.27 (hCV9701001), Chr6.29 (hCV2461889), Chr6.30 (2461901), and Chr6.31 (hCV2461981). Those TaqMan probes and their locations on chromosome 6 are shown in FIG. 10. Each different TaqMan assay quantitated amplification products at a different position along Chromosome 6. Thus, amplification products from each amplification reaction were evaluated at 24 individual positions along the length of chromosome 6. The results of the chromosomal amplification bias assays for each of the seven primers are shown in FIG. 11. The amplification profiles of primer sets DR03, DR03B, DR07B, and DR15B are graphically represented in FIG. 12.

### EXAMPLE 11

Amplification Analysis Using a Third Series of Primer Sets

Twenty six different primer sets were individually tested for their ability to amplify nucleic acids in an amplification reaction. One primer set was used for each different amplification reaction. The twenty six primer sets that were used appear in Table 3 below.

TABLE 3

| PRIMER SET | SEQUENCE |
|---|---|
| DR1C | CTNNNNNNTC |
| DR2C | CTNNNNNTC |
| DR3C | CTNNNNNNYY |
| DR4C | CTCNNNNYY |
| DR5C | YYNNNNNNTC |
| DR6C | YYNNNNTC |
| DR7C | YYNNNNYY |
| DR8C | YYYYYYYYYY |
| DR9C | YYYYYYYY |
| DR10C | RRRRRRRRRR |
| DR11C | RRRRRRRR |
| DR12C | GANNNNNNNR |
| DR13C | GANNNNNR |
| DR14C | GANNNNNNRR |
| DR15C | GANNNNRR |
| DR16C | NNNNTTTNNN |
| DR17C | NNNYYYYNNN |
| DR18C | NNYYYYNN |
| DR19C | NNYYYYYYNN |
| DR20C | NYYYYYYN |
| DR21C | TTAGGGNN |
| DR22C | NNTTAGGGNN |
| DR23C | NNTTAGGG |
| DR24C | CCGATCGC |
| DR25C | ACGATCGG |
| DR26C | NCGATCGN |

Amplification reactions were performed as follows:

Separate solutions of solution A were prepared by adding 5 μl of a 500 μM stock of one of primer sets DR1C to DR26C, 2 μl of neat DMSO (American Type Culture Collection (ATCC), Rockville, Md.), 0.5 μl of 100 mM dNTPs (25 mM each: dATP, dCTP, dGTP and dTTP) (Applied Biosystems, Foster City, Calif.), 4 μl of 10× Thermopol buffer (New England Biolabs, Beverly, Mass.), and an amount of human genomic DNA as set forth below, to a final solution volume of 40 μl. For each primer set, one solution A was formed comprising 0 ng of Human genomic DNA template (Clontech, Palo Alto, Calif.); and one solution A was formed comprising 10 ng of Human genomic DNA template (Clontech, Palo Alto, Calif.).

Solution B was prepared by adding 2.2 μl of Bst DNA Polymerase Large-Fragment stock enzyme (New England Biolabs, Beverly, Mass. (NEB catalog #M0275L, 8,000 Units/ml)) and 1.0 μl of 10× Thermopol buffer (New England Biolabs, Beverly, Mass.) to a final volume of 10 μl.

Solution A was added into a well of a 96-well thermocycler plate, such that there were 52 wells comprising a solution A in the 96 well plate, each well comprising a different primer set. The plate wells were sealed, and the plate was placed into an ABI 9700 thermocycler (Applied Biosystems, Foster City, Calif.). The plate was heated at 94° C. for 3 minutes. Following the heating step, the plate was cooled to 4° C. for 1 to 5 minutes. The plate was then heated to 50° C. and the wells were unsealed. Once the wells were unsealed, 10 μl of solution B was added with gentle mixing. The wells were resealed, and the plate was maintained at 50° C. for 5 hours.

The reaction composition in each well comprised the following:

1× ThermoPol (20 mM Tris-HCl pH 8.8, 10 mM KCl, 10 mM (NH₄)SO₄, 2 mM MgSO₄, 0.1% Triton X-100)

4% neat DMSO

50 μM Primer

1 mM dNTPs (0.25 mM each: dATP, dCTP, dGTP and dTTP)

0.35 U/μl Bst DNA Polymerase (Large-Fragment)

Human genomic DNA template (0 ng or 10 ng) (Clontech, Palo Alto, Calif.)

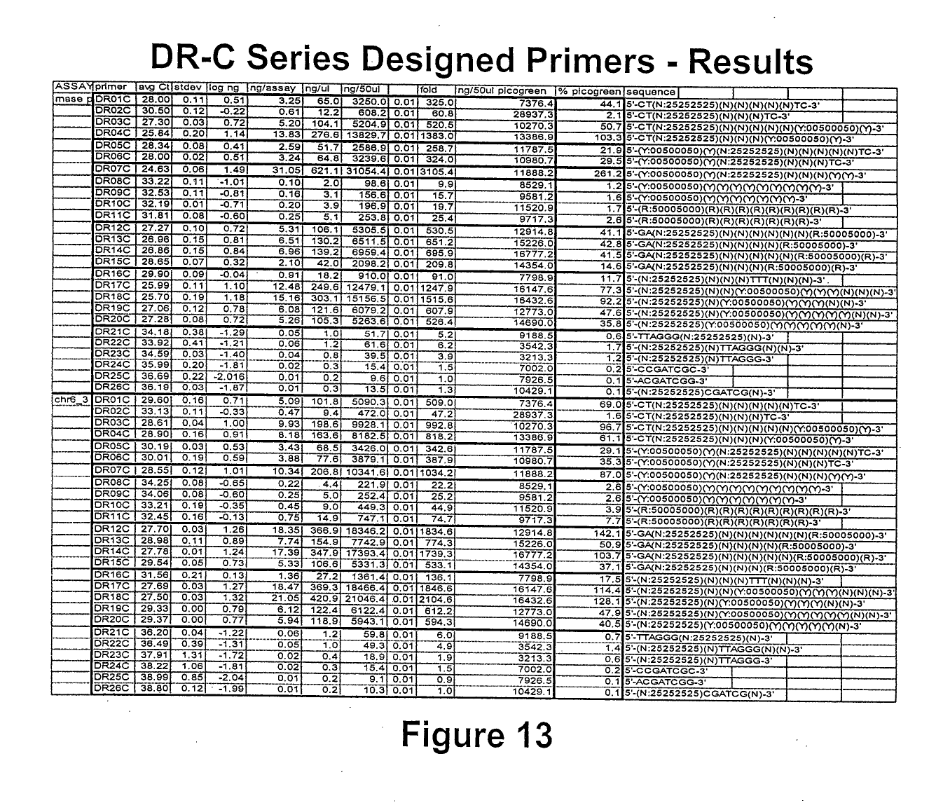

The reaction products from each amplification reaction were analyzed using a picogreen assay and two TaqMan assays. Picogreen assays were carried out as described in Example 3. The TaqMan assays were performed at two positions and were carried out as described in Example 4. The two TaqMan probes were the RNase P probe, which is described in Example 4, and the Chr6.3 (hCV2498203) probe, which is described in Example 10 and FIG. 10. The results of those assays are shown in FIG. 13.

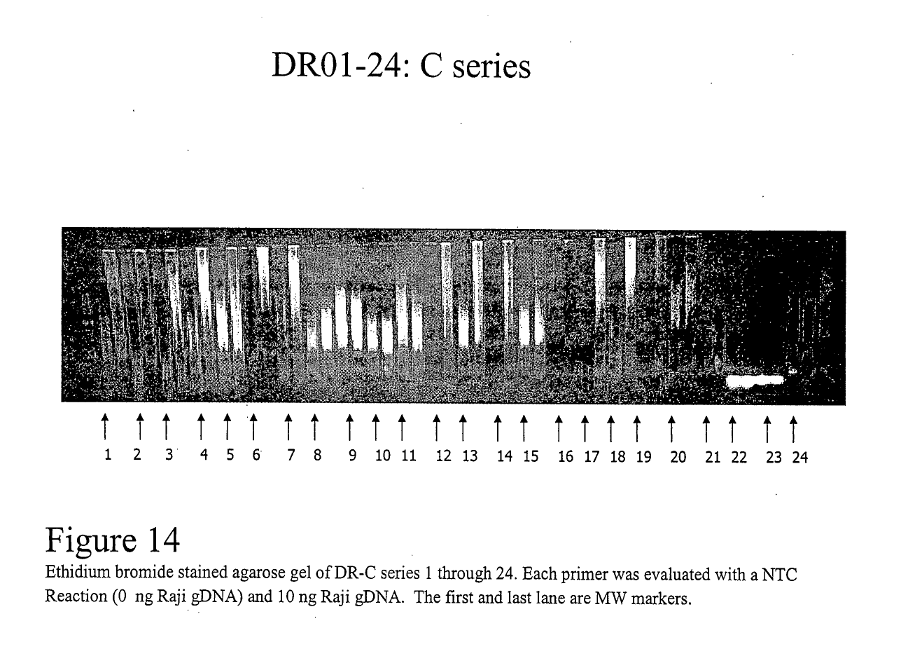

The products of each amplification reaction were also analyzed using agarose gel electrophoresis as described in Example 2. Reaction products were subjected to electrophoresis on an agarose gel as is shown in FIG. 14.

### EXAMPLE 12

Amplification Analysis Using a Fourth Series of Primer Sets

Fourteen different primer sets were individually tested for their ability to amplify nucleic acids in an amplification reaction. One primer set was used for each different amplification reaction. The fourteen primer sets that were used appear in Table 4 below.

TABLE 4

| PRIMER SET | SEQUENCE |
|---|---|
| DR01D | TTNNNNNNNN |
| DR02D | CCNNNNNNNN |
| DR03D | GGNNNNNNNN |
| DR04D | AANNNNNNNN |
| DR05D | NNNNNNNNTT |
| DR06D | NNNNNNNNCC |
| DR07D | NNNNNNNNGG |
| DR08D | NNNNNNNNAA |
| DR09D | WWNNNNNNNN |
| DR10D | SSNNNNNNNN |
| DR11D | NNNNNNNNWW |
| DR12D | NNNNNNNNSS |
| DR13D | NNNWWWNNN |
| DR14D | NNNSSSNNN |

Amplification reactions were performed as follows:

Separate solutions of solution A were prepared by adding 5 μl of a 500 μM stock of one of primer sets DR1D to DR14D, 2 μl of neat DMSO (American Type Culture Collection (ATCC), Rockville, Md.), 0.5 μl of 100 mM dNTPs (25 mM each: dATP, dCTP, dGTP and dTTP) (Applied Biosystems, Foster City, Calif.), 4 μl of 10× Thermopol buffer (New England Biolabs, Beverly, Mass.), and an amount of human genomic DNA as set forth below, to a final solution volume of 40 μl. For each primer set, one solution A was formed comprising 0 ng of Human genomic DNA template (Clontech, Palo Alto, Calif.); and one solution A was formed comprising 10 ng of Human genomic DNA template (Clontech, Palo Alto, Calif.).

Solution B was prepared by adding 2.2 μl of Bst DNA Polymerase Large-Fragment stock enzyme (New England Biolabs, Beverly, Mass. (NEB catalog #M0275L, 8,000 Units/ml)) and 1.0 μl of 10× Thermopol buffer (New England Biolabs, Beverly, Mass.) to a final volume of 10 μl.

Solution A was added into a well of a 96-well thermocycler plate, such that there were 28 wells comprising a solution A in the 96 well plate, each well comprising a different primer set. The plate wells were sealed, and the plate was placed into an ABI 9700 thermocycler (Applied Biosystems, Foster City, Calif.). The plate was heated at 94° C. for 3 minutes. Following the heating step, the plate was cooled to 4° C. for 1 to 5 minutes. The plate was then heated to 50° C. and the wells were unsealed. Once the wells were unsealed, 10 μl of solution B was added with gentle mixing. The wells were resealed, and the plate was maintained at 50° C. for 5 hours.

The reaction composition in each well comprised the following:

1× ThermoPol (20 mM Tris-HCl pH 8.8, 10 mM KCl, 10 mM (NH₄)SO₄, 2 mM MgSO₄, 0.1% Triton X-100)

4% neat DMSO

50 μM Primer

1 mM dNTPs (0.25 mM each: dATP, dCTP, dGTP and dTTP)

0.35 U/μl Bst DNA Polymerase (Large-Fragment)

Human genomic DNA template (0 ng or 10 ng) (Clontech, Palo Alto, Calif.)

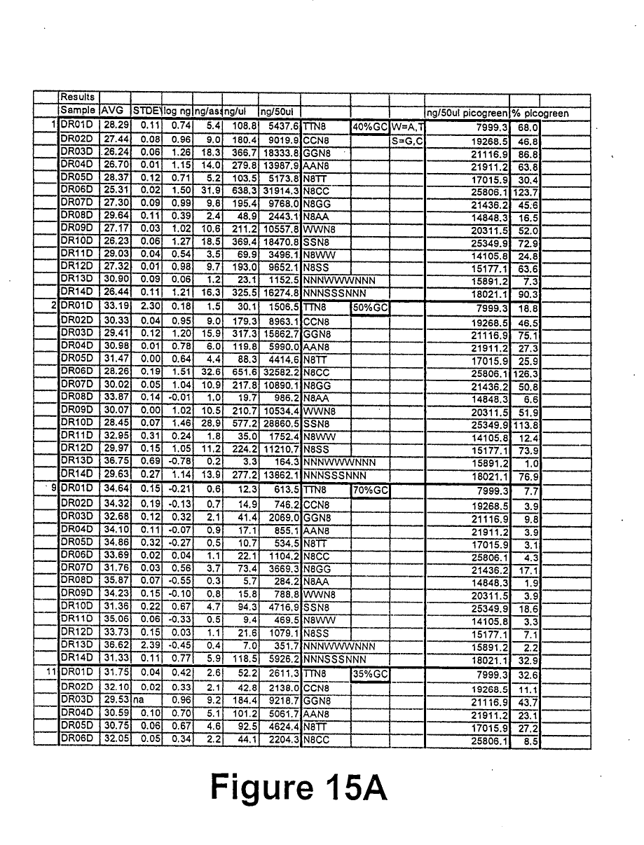

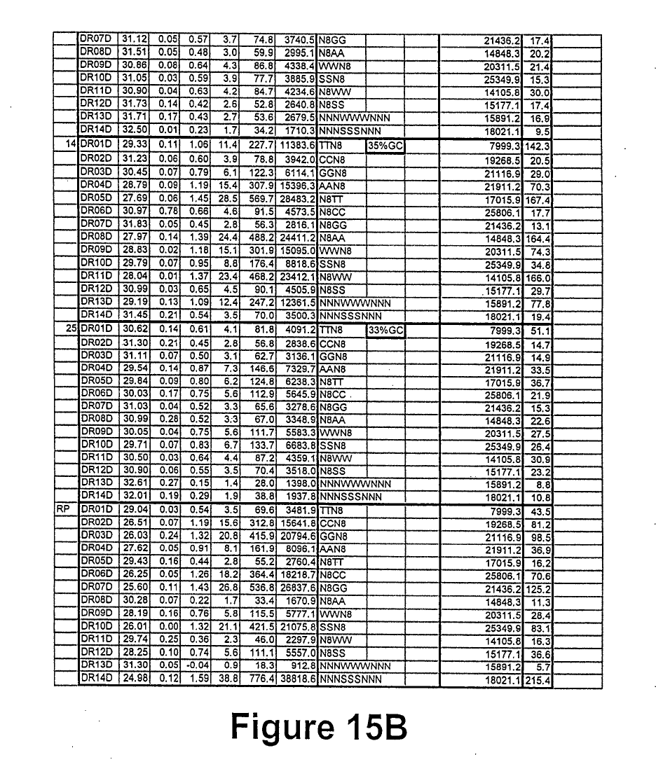

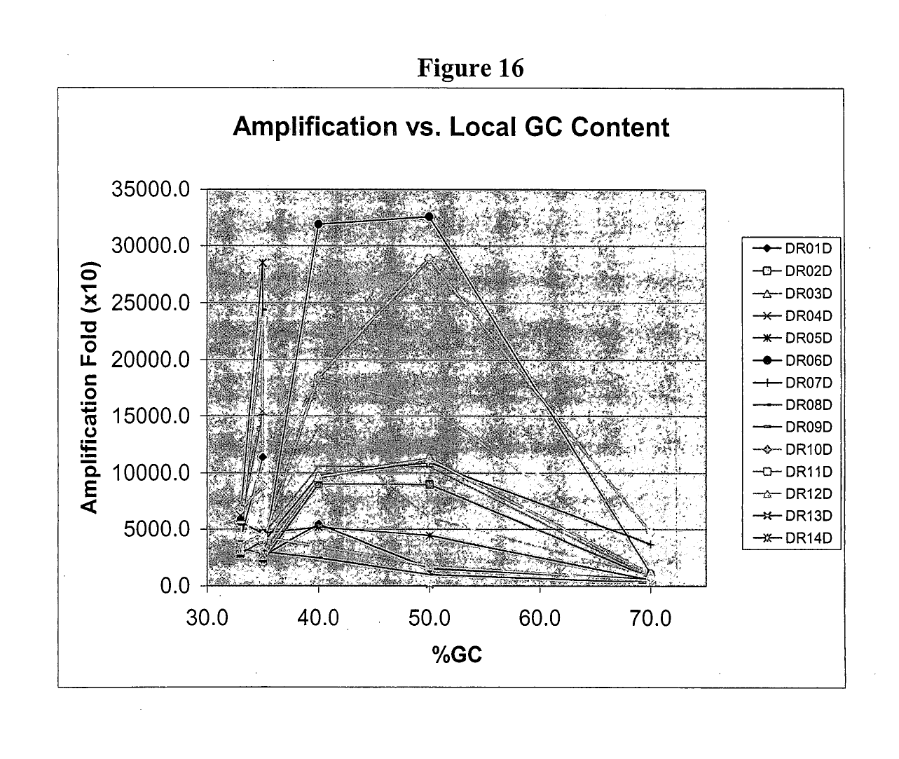

The amplification products for each primer set were measured for amplification of genomic DNA with variable GC content as follows. A set of TaqMan assays were used to evaluate the amplification of six positions on Human Chromosome 6. The six positions for evaluation were selected to measure amplification bias associated with amplifying regions with different GC content. The assay designated 1 corresponds to TaqMan assay Chr6.1 (hCV2498239) in FIG. 10. TaqMan assay Chr6.1 measures amplification at a position where the GC content was 40% GC. The assay designated 2 corresponds to TaqMan assay Chr6.2 (hCV2498215) in FIG. 10. TaqMan assay Chr6.2 measures amplification at a position where the GC content is 50% GC. The assay designated 9 corresponds to TaqMan assay Chr6.9 (hCV1478558) in FIG. 10. TaqMan assay Chr6.9 measures amplification at a position where the GC content is 70% GC. The assay designated 11 corresponds to TaqMan assay Chr6.11 (hCV8650583) in FIG. 10. TaqMan assay Chr6.11 measures amplification at a position where the GC content is 35% GC. The assay designated 14 corresponds to TaqMan assay Chr6.14 (hCV1820235) in FIG. 10. TaqMan assay Chr6.14 measures amplification at a position where the GC content is 35% GC. The assay designated 25 corresponds to TaqMan assay Chr6.25 (hCV1361933) in FIG. 10. TaqMan assay Chr6.25 measures amplification at a position where the GC content is 33% GC. The assay designated RP corresponds to the RNase P TaqMan assay described in Example 4. The GC content was not calculated for the position to which RNase P Taqman probe hybridizes. GC content for the genomic region surrounding the position that hybridizes to the TaqMan probe of a particular assay was estimated from the NCBI dbSNP sequence entry corresponding to that assay. The results of those assays are shown in FIG. 15. The fold amplification compared to GC content is shown in FIG. 16.

### EXAMPLE 13

Amplification Analysis Using a Fifth Series of Primer Sets

Eighty different primer sets were individually tested for their ability to amplify nucleic acids in an amplification reaction. One primer set was used for each different amplification reaction. The eighty primer sets that were used appear in Table 5 below.

TABLE 5

| PRIMER SET | SEQUENCE |
|---|---|
| DR1E | NNNSSNSSS |
| DR2E | NNSNSSNSNN |
| DR3E | SNSNSSNSNS |
| DR4E | NSNSSSNSN |
| DR5E | NNNSSSNNS |
| DR6E | NNNSSSNSS |
| DR7E | SNNSSSNNN |
| DR8E | SSNSSSNNN |
| DR9E | NNSSSSNNN |
| DR10E | NNNSSSSNN |
| DR11E | NNNSSSSNNS |
| DR12E | SNNSSSSNNN |
| DR13E | SSNNNNNNSS |
| DR14E | SSNNNNNSS |
| DR15E | SSSNNNSSS |
| DR16E | SSSNNNNSSS |
| DR17E | SSSNNNNSS |
| DR18E | SSNNNNSSS |
| DR19E | NNNNNNNSSS |
| DR20E | SSSNNNNNNN |
| DR21E | NNNNNNSSSS |
| DR22E | SSSSNNNNNN |
| DR23E | SSSSSSSS |
| DR24E | SSSSSSSSSS |
| DR25E | YYNNNNSS |
| DR26E | SSNNNNYY |
| DR27E | YSNNNNYY |
| DR28E | SYNNNNYY |
| DR29E | YYNNNNSY |
| DR30E | YYNNNNYS |
| DR31E | SYNNNNSY |
| DR32E | SYNNNNYS |
| DR33E | YSNNNNYS |
| DR34E | YSNNNNSY |
| DR35E | YYNNNNNNSS |
| DR36E | SSNNNNNNYY |
| DR37E | YSNNNNNNYY |
| DR38E | SYNNNNNNYY |
| DR39E | YYNNNNNNSY |
| DR40E | YYNNNNNNYS |
| DR41E | SYNNNNNNSY |
| DR42E | SYNNNNNNYS |
| DR43E | YSNNNNNNYS |
| DR44E | YSNNNNNNSY |
| DR45E | NNNYYYNNN |
| DR46E | NNNYYYNNNN |
| DR47E | NNNNYYYNNN |
| DR48E | NNNNNYYYNN |
| DR49E | NNNNNNYYYN |
| DR50E | NNNNNNNYYY |
| DR51E | YYYNNNNNNN |
| DR52E | NYYYNNNNNN |
| DR53E | NNYYYNNNNN |
| DR54E | NNNNSSSNN |
| DR55E | NNNNNSSSN |
| DR56E | NNNNNNSSS |
| DR57E | SSSNNNNNN |
| DR58E | NSSSNNNNN |
| DR59E | NNSSSNNNN |
| DR60E | NNNNSSSNNN |
| DR61E | NNNNNSSSNN |
| DR62E | NNNNNNSSSN |
| DR63E | NSSSNNNNNN |
| DR64E | NNSSSNNNNN |
| DR65E | NNNSSSNNNN |
| DR66E | NWNNNNNNSS |
| DR67E | NNNNWWWWSS |
| DR68E | NNNWWWWSS |
| DR69E | NNNWWWNSS |
| DR70E | NNNWWNNSS |
| DR71E | WWNNNNNSS |
| DR72E | CTNNNNNNNS |
| DR73E | CTNNNNNNSS |
| DR74E | CTNNNNNSSS |
| DR75E | CTNNNNNYY |
| DR76E | CTNNNNNNY |
| DR77E | CTNNSSNNNN |
| DR78E | CTNSSSNNNN |
| DR79E | CTNNSSSNNN |
| DR80E | CTNNYYNNNN |

Each primer set was tested in four different amplification reactions. The first reaction used 2× Bst DNA polymerase with human genomic DNA and the second reaction was a No Template Control reaction with 2× Bst DNA polymerase. The third reaction used 1.5× Bst DNA polymerase with human genomic DNA and 1M Betaine; and the fourth reaction was a No Template Control reaction with 1.5× Bst DNA polymerase and 1M Betaine.

Separate solutions of solution A were prepared by adding 5 μl of a 500 μM stock of one of primer sets DR1E to DR80E, 2 μl of neat DMSO (American Type Culture Collection (ATCC), Rockville, Md.), 0.5 μl of 100 mM dNTPs (25 mM each: dATP, dCTP, dGTP and dTTP)(Applied Biosystems, Foster City, Calif.), and 4 μl of 10× Thermopol buffer (New England Biolabs, Beverly, Mass.), and an amount of human genomic DNA and Betaine as set forth below, to a final solution volume of 40 μl. The amount of Human genomic DNA template added was either 1 μl or 0 μl of a 10 ng/μl stock of Human genomic DNA template (Clontech, Palo Alto, Calif.) depending on whether the reaction composition contained human genomic DNA or was a No Template Control, respectively. The amount of Betaine added was either 0 μl or 10 μl of 5M Betaine (Sigma-Aldrich, St. Louis, Mo.) depending on whether the reaction composition was a 2× Bst reaction or a 1.5× Bst reaction, respectively.

Solution B was prepared by adding Bst DNA Polymerase Large-Fragment stock enzyme (New England Biolabs, Beverly, Mass. (NEB catalog #M0275L, 8,000 Units/ml)) and 1.0 μl of 10× Thermopol buffer (New England Biolabs, Beverly, Mass.) to a final volume of 10 μl. The amount of Bst DNA polymerase added was either 4.4 μl or 3.3 μl depending on whether the reaction was a 2× Bst reaction or a 1.5× Bst reaction, respectively

Solution A was added into a well of a 96-well thermocycler plate, such that there were 320 wells distributed across four 96 well plates, each of the 320 wells comprising a solution A, and each of those wells comprising a different primer set. The plate wells were sealed, and the plate was placed into an ABI 9700 thermocycler (Applied Biosystems, Foster City, Calif.). The plate was heated at 94° C. for 3 minutes. Following the heating step, the plate was cooled to 4° C. for 1 to 5 minutes. The plate was then heated to 50° C. and the wells were unsealed. Once the wells were unsealed, 10 μl of solution B was added with gentle mixing. The wells were resealed, and the plate was maintained at 50° C. for 5 hours for the 2× Bst DNA polymerase reactions. For the 1.5× Bst DNA polymerase reactions, the plate was maintained at 47° C. for 16 hours.

For the reaction composition with 2× Bst DNA polymerase, the reaction composition in each well comprised the following:

1× ThermoPol (20 mM Tris-HCl pH 8.8, 10 mM KCl, 10 mM (NH₄)SO₄, 2 mM MgSO₄, 0.1% Triton X-100)

4% neat DMSO

50 μM Primer

1 mM dNTPs (0.25 mM each: dATP, dCTP, dGTP and dTTP)

0.7 U/μl Bst DNA Polymerase (Large-Fragment)

Human genomic DNA template (0 ng or 10 ng) (Clontech, Palo Alto, Calif.)

For the reaction composition with 1.5× Bst DNA, the reaction composition in each well comprised the following:

1× ThermoPol (20 mM Tris-HCl pH 8.8, 10 mM KCl, 10 mM (NH₄)SO₄, 2 mM MgSO₄, 0.1% Triton X-100)

4% neat DMSO

50 μM Primer

1 mM dNTPs (0.25 mM each: dATP, dCTP, dGTP and dTTP)

0.53 U/μl Bst DNA Polymerase (Large-Fragment)

1M Betaine

Human genomic DNA template (0 ng or 10 ng) (Clontech, Palo Alto, Calif.)

The amplification products from each amplification reaction were measured using four different TaqMan probes. The TaqMan assays were performed as described in Example 4. The four TaqMan probes that were used were the RNaseP probe, the Chr6.9 probe, the Chr6.2 probe, and the Chr6.14 probe. The RNaseP probe was previously described at Example 4. The Chromosome 6.9 probe, Chromosome 6.2 probe and Chromosome 6.14 probe are all described in Example 10 and FIG. 10.

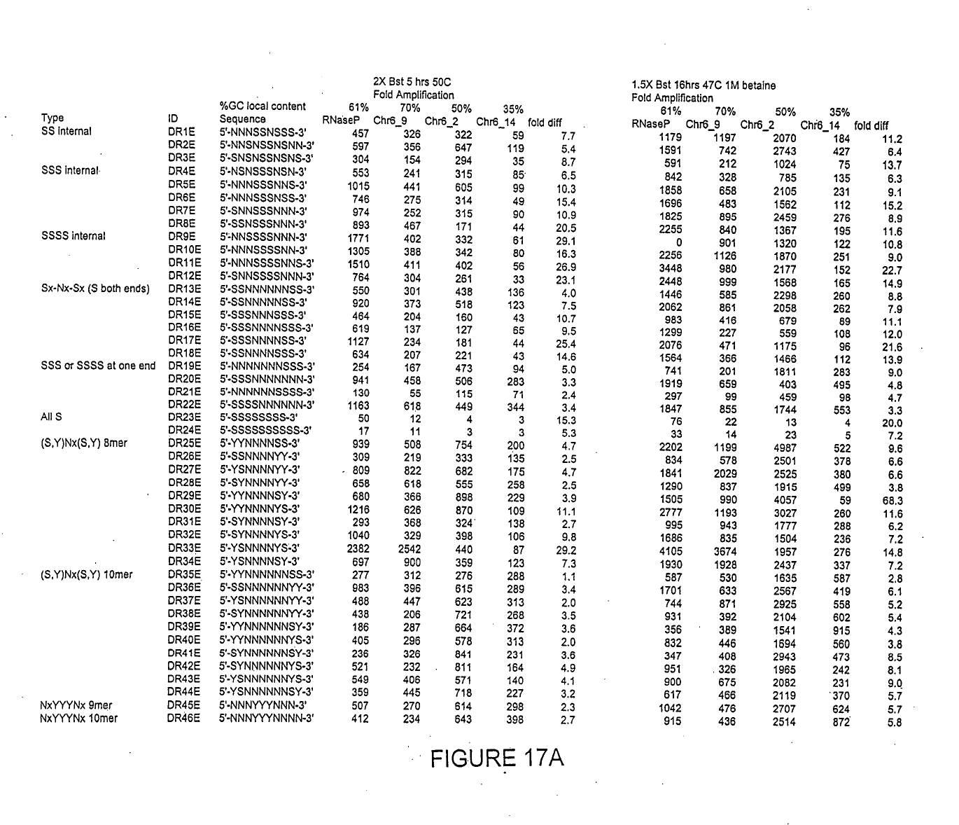

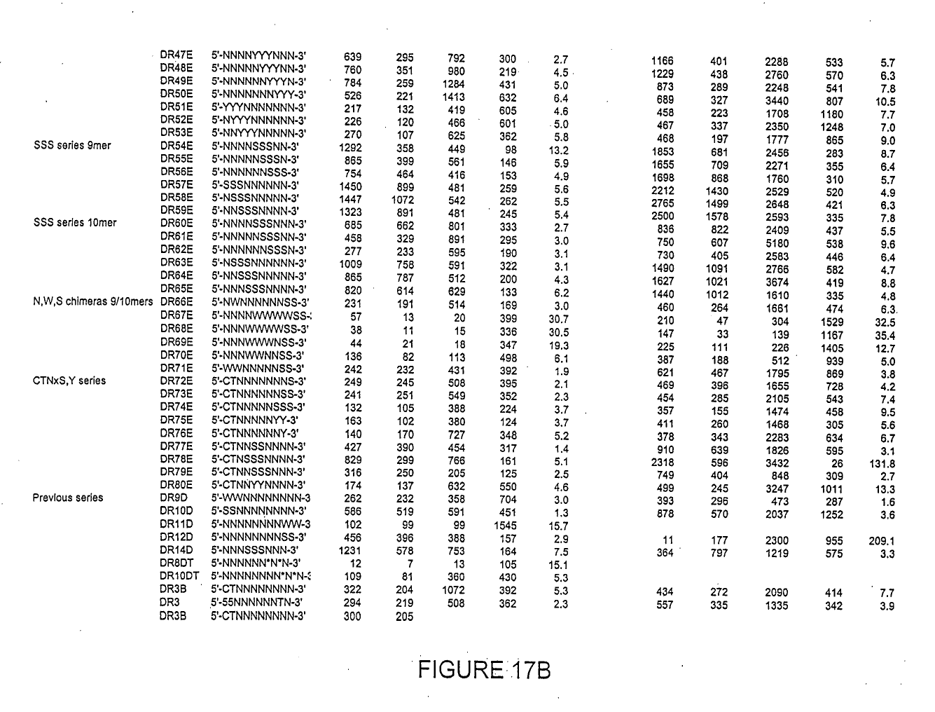

The results of the different amplification reactions are shown in FIG. 17. That figure shows fold amplification results for 4 TaqMan assays with different local GC contents and the inter-locus fold differences in amplification with the different primers under the two experimental conditions.

### EXAMPLE 14

Amplification Analysis Using a Sixth Series of Primer Sets

Sixty three different primer sets were individually tested for their ability to amplify nucleic acids in an amplification reaction. One primer set was used for each different amplification reaction. The sixty three primer sets that were used appear in Table 6 below.

TABLE 6

| PRIMER SET | SEQUENCE |
|---|---|
| DR1F | NNSSSNNNWW |
| DR2F | NSSSNNNNWW |
| DR3F | SSSNNNNNWW |
| DR4F | NNSSSWWWSS |
| DR5F | NSSSNWWWSS |
| DR6F | SSSNNWWWSS |
| DR7F | NNSSSNWWSS |
| DR8F | NNSSSWWNSS |
| DR9F | SSSNNNWWSS |
| DR10F | NSSSNNWWSS |
| DR11F | NNSSSNWWSS |
| DR14F | NSSSNWWNSS |
| DR15F | SSSNNWWNSS |
| DR16F | SSSNWWNNSS |
| DR17F | SSNNNNNNWW |
| DR18F | SSNNNNNWWN |
| DR19F | SSNNNNWWNN |
| DR20F | SSNNNWWNNN |
| DR21F | SSNNWWNNNN |
| DR22F | SSNNNNNWWW |
| DR23F | SSNNNNWWWN |
| DR24F | SSNNNWWWNN |
| DR25F | SSNNWWWNNN |
| DR26F | SSNWWWNNNN |
| DR27F | YNNNNNSS |
| DR28F | YYYNNNSS |
| DR29F | WWNNNNSS |
| DR30F | WWWBBBSS |
| DR31F | WWWNNNSS |
| DR32F | YBNNNNSS |
| DR33F | YBBNNNSS |
| DR34F | BYNNNNSS |
| DR35F | WWBNNNSS |
| DR36F | WWBBNNSS |
| DR37F | BBNNNNSS |
| DR38F | SSNNNNNYY |
| DR39F | SSNNNNWYY |
| DR40F | SSNNNWNYY |
| DR41F | SSNNWNNYY |
| DR42F | SSNWNNNYY |
| DR42F(2) | SSWNNNNYY |
| DR43F | YSNNNNNYS |
| DR44F | YSNNNNWYS |
| DR45F | YSNNWNNYS |
| DR46F | YSNWNNNYS |
| DR47F | YSWNNNNYS |
| DR48F | SSNNNNYY |
| DR49F | SSNNNWYY |
| DR50F | SSNNWNYY |
| DR51F | SSNWNNYY |
| DR52F | SSWNNNYY |
| DR53F | YYNNNNNSS |
| DR54F | SSNNNNNYY |
| DR55F | YSNNNNNYY |
| DR56F | SYNNNNNYY |
| DR57F | YYNNNNNSY |
| DR58F | YYNNNNNYS |
| DR59F | SYNNNNNSY |
| DR60F | SYNNNNNYS |
| DR61F | YSNNNNNYS |
| DR62F | YSNNNNNSY |

Amplification reactions were performed as follows:

Separate solutions of solution A were prepared by adding 5 μl of a 500 μM stock of one of primer sets DR01F to DR62F, 2 μl of neat DMSO (American Type Culture Collection (ATCC), Rockville, Md.), 0.5 μl of 100 mM dNTPs (25 mM each: dATP, dCTP, dGTP and dTTP) (Applied Biosystems, Foster City, Calif.), 4 μl of 10×Thermopol buffer (New England Biolabs, Beverly, Mass.), and 10 ng of Human genomic DNA template (Clontech, Palo Alto, Calif.) to a final solution volume of 40 μl.

Solution B was prepared by adding 2.2 μl of Bst DNA Polymerase Large-Fragment stock enzyme (New England Biolabs, Beverly, Mass. (NEB catalog #M0275L, 8,000 Units/ml)) and 1.0 μl of 10× Thermopol buffer (New England Biolabs, Beverly, Mass.) to a final volume of 10 μl.

Solution A was added into a well of a 96-well thermocycler plate, such that there were 63 wells comprising a solution A in the 96 well plate, each well comprising a different primer set. The plate wells were sealed, and the plate was placed into an ABI 9700 thermocycler (Applied Biosystems, Foster City, Calif.). The plate was heated at 94° C. for 3 minutes. Following the heating step, the plate was cooled to 4° C. for 1 to 5 minutes. The plate was then heated to 50° C. and the wells were unsealed. Once the wells were unsealed, 10 μl of solution B was added with gentle mixing. The wells were resealed, and the plate was maintained at 50° C. for 5 hours.

The reaction composition in each well comprised the following:

1× ThermoPol (20 mM Tris-HCl pH 8.8, 10 mM KCl, 10 mM (NH₄)SO₄, 2 mM MgSO₄, 0.1% Triton X-100)

4% neat DMSO

50 μM Primer

1 mM dNTPs (0.25 mM each: dATP, dCTP, dGTP and dTTP)

0.35 U/μl Bst DNA Polymerase (Large-Fragment)

Human genomic DNA template (10 ng) (Clontech, Palo Alto, Calif.)

The amplification products from each amplification reaction were measured using eight different TaqMan probes. The TaqMan assays were performed as described in Example 4. The eight TaqMan probes that were used are the RNaseP probe, the Chr6.9 probe, the Chr6.2 probe, the Chr6.14 probe, the Chr6.1 probe, the Chr6.11 probe, the Chr6.24 probe, and the Chr6.25 probe. The RNaseP probe was previously described in Example 4. The Chromosome 6.9 probe, Chromosome 6.2 probe, Chromosome 6.14 probe, Chromosome 6.1 probe, Chromosome 6.11 probe, Chromosome 6.24 probe, and Chromosome 6.25 probe are all described in Example 10 and FIG. 10.

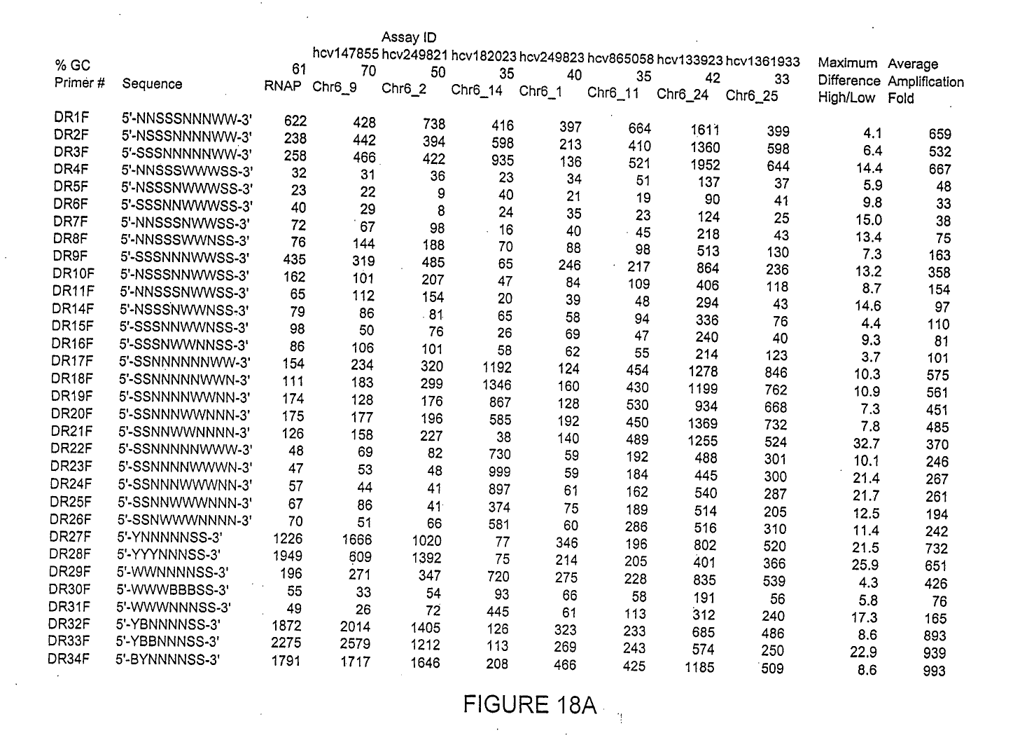

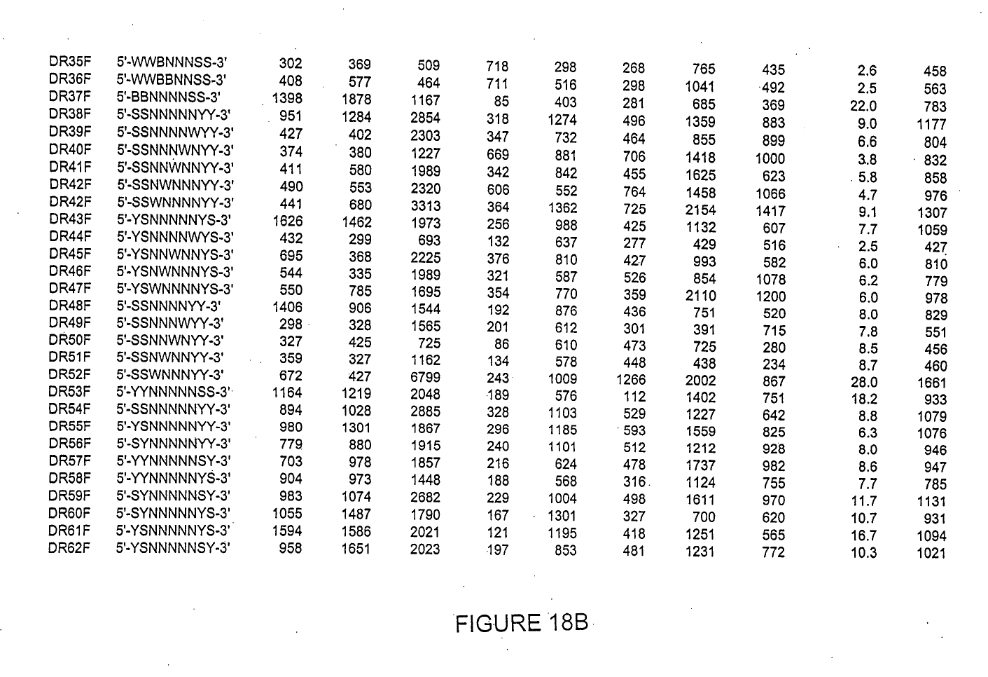

The results of the different amplification reactions are shown in FIG. 18.

### EXAMPLE 15

Amplification of a cDNA Library

cDNA was prepared from human liver total RNA (Clontech catalog # 640221-1). The cDNA was prepared using the Applied Biosystems High-Capacity cDNA Archive Kit (catalog # 4322171) using two priming schemes. The first priming scheme used random primers (Applied Biosystems, Foster City, Calif.) at a 20 μM final reaction concentration. The second priming scheme used Oligo dT₁₆ (Applied Biosystems, Foster City, Calif.) at a 5 μM final reaction concentration. The cDNA reaction contained 100 ng/μl of total RNA. Otherwise, the cDNAs were prepared as described in the Applied Biosystems High Capacity cDNA Archive Kit.

Six different reaction compositions were prepared as follows:

Six separate solutions of solution A were prepared by adding 5 μl of a 500 μM stock of one of primer sets N₈, N₁₀, or DR10D (SSN₈) as discussed below, 2 μl of neat DMSO (American Type Culture Collection (ATCC), Rockville, Md.), 0.5 μl of 100 mM dNTPs (25 mM each: dATP, dCTP, dGTP and dTTP) (Applied Biosystems, Foster City, Calif.), and 4 μl of 10× Thermopol buffer (New England Biolabs, Beverly, Mass.), and cDNA as discussed below, to a final solution volume of 40 μl.

The first solution A, the second solution A, and the third solution A each comprised 10 ng of cDNA formed from the random primer priming scheme. The first solution A also comprised the primer set N₈. The second solution A also comprised the primer set N₁₀. The third solution A also comprised the primer set DR10D (SSN₈).

The fourth solution A, the fifth solution A, and the sixth solution A each comprised 10 ng of cDNA formed from the Oligo dT₁₆ priming scheme. The fourth solution A also comprised the primer set N₈. The fifth solution A also comprised the primer set N₁₀. The sixth solution A also comprised the primer set DR10D (SSN₈).

Solution B was prepared by adding 2.2 μl of Bst DNA Polymerase Large-Fragment stock enzyme (New England Biolabs, Beverly, Mass. (NEB catalog #M0275L, 8,000 Units/ml)) and 1.0 μl of 10× Thermopol buffer (New England Biolabs, Beverly, Mass.) to a final volume of 10 μl.

Solution A was added into a well of a 96-well thermocycler plate, such that there were 28 wells comprising a solution A in the 96 well plate, each well comprising a different primer set. The plate wells were sealed, and the plate was placed into an ABI 9700 thermocycler (Applied Biosystems, Foster City, Calif.). The plate was heated at 94° C. for 3 minutes. Following the heating step, the plate was cooled to 4° C. for 1 to 5 minutes. The plate was then heated to 50° C. and the wells were unsealed. Once the wells were unsealed, 10 μl of solution B was added with gentle mixing. The wells were resealed, and the plate was maintained at 50° C. for 5 hours.

The reaction composition in each well comprised the following:

1× ThermoPol (20 mM Tris-HCl pH 8.8, 10 mM KCl, 10 mM (NH₄)SO₄, 2 mM MgSO₄, 0.1% Triton X-100)

4% neat DMSO

50 μM Primer

1 mM dNTPs (0.25 mM each: dATP, dCTP, dGTP and dTTP)

0.35 U/μl Bst DNA Polymerase (Large-Fragment)

cDNA from either the random primer scheme or from the Oligo dT₁₆ scheme (10 ng)

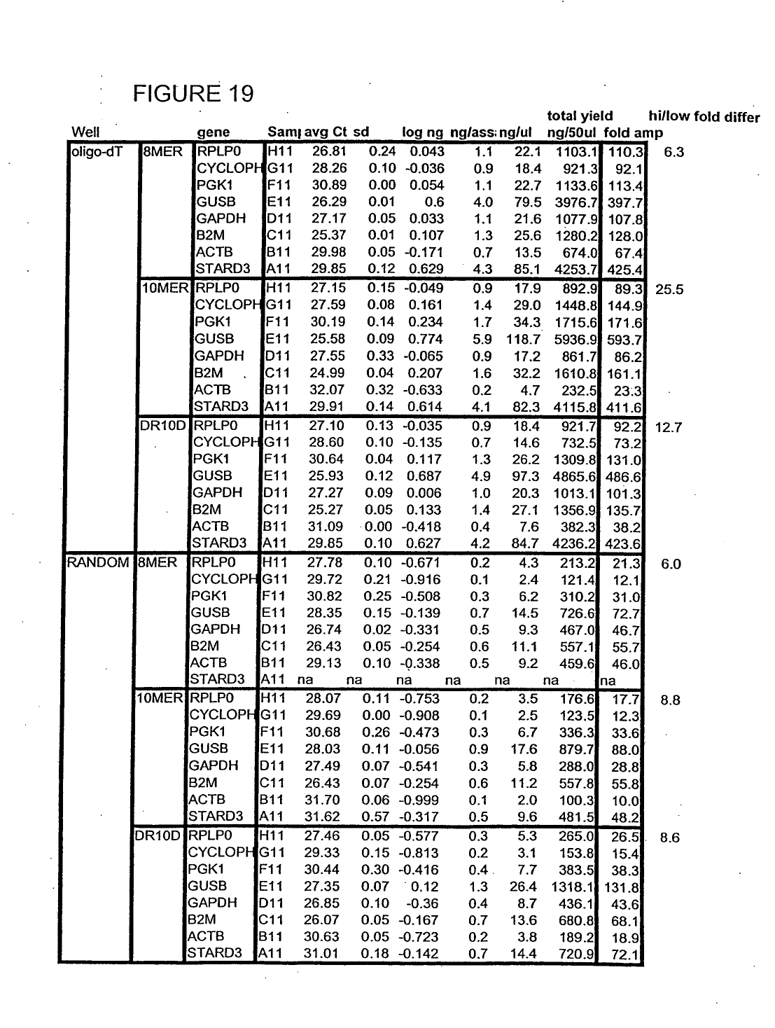

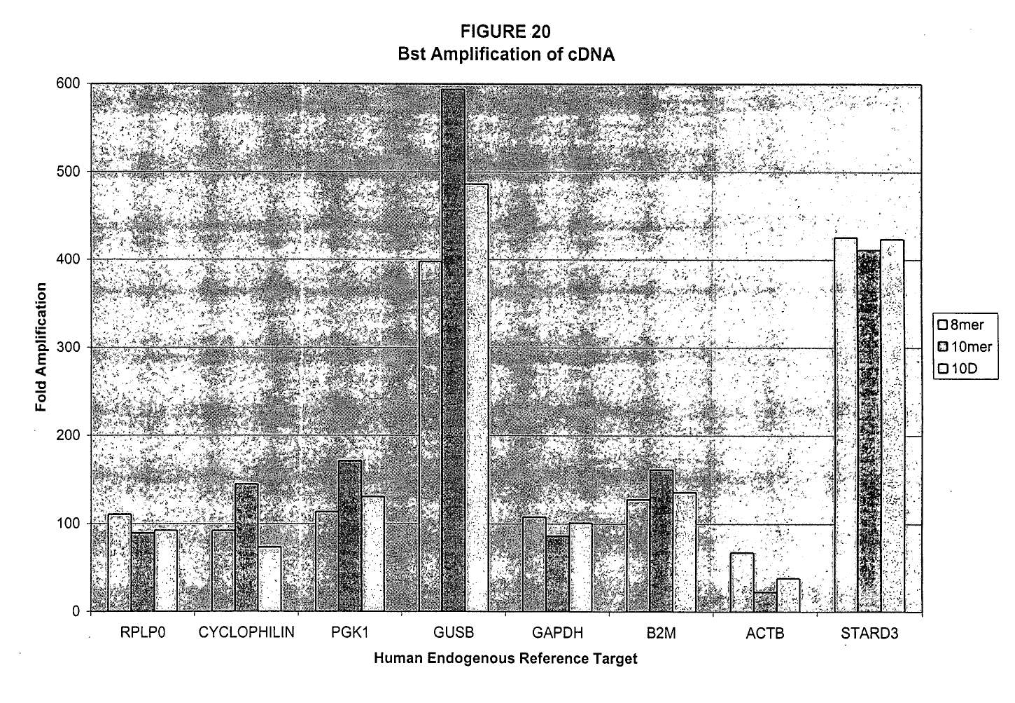

Amplification products from the cDNA prepared with random-primers were compared to amplification products from the cDNA prepared with Oligo dT₁₆. TaqMan assays were used to measure amplification as described in Example 4. TaqMan probes were used to evaluate the amplification of eight different cDNAs-RPLP0, Cyclophilin, PGK1, GUSB, GAPDH, B2M, ACTB, and STARD3. The TaqMan probes for these genes were hs99999902_m1, hs99999904_m1, hs99999906_m1, hs99999908_m1, hs99999905_m1, hs99999907_m1, hs99999903_m1, and hs00199052_m1, respectively. (Those numbers correspond to catalog numbers of assays available from Applied Biosystems, Foster City, Calif.). Those results are shown in FIG. 19. The results of amplification of a cDNA library prepared with Oligo dT₁₆ are graphed in FIG. 20.

### EXAMPLE 16

Amplification of Nucleic Acids from Bacteria (Bacillus ceres)

Amplification reactions were used to amplify genomic DNA from bacteria. A 500 μl overnight culture of Bacillus ceres was microcentrifuged for 1 minute to pellet the bacterial cells. The bacterial cell pellet was resuspended in a 10 μl 1N NaOH lysis solution. After 2 hours at room temperature, the lysis solution was neutralized by adding 10 μl 1M Tris-HCl 8.0 with gentle mixing. For a no template control, 50 ul of TE was stored on ice.

Four different reaction compositions were prepared as follows:

Four separate solutions of solution A were prepared by adding 5 μl of a 500 μM stock of primer set DR10D (SSN₈), 2 μl of neat DMSO (American Type Culture Collection (ATCC), Rockville, Md.), 0.5 μl of 100 mM dNTPs (25 mM each: dATP, dCTP, dGTP and dTTP) (Applied Biosystems, Foster City, Calif.), 4 μl of 10× Thermopol buffer (New England Biolabs, Beverly, Mass.), and an amount of B. ceres lysate as discussed below, to a final solution volume of 40 μl. The first solution A comprised the no template control; the second solution A comprised 0.01 μl of equivalent B. ceres lysate (1 ul of a 1:100 dilution); the third solution A comprised 0.1 μl of B. ceres lysate; and the fourth solution A comprised 1.0 μl of B. ceres lysate.

Solution B was prepared by adding 2.2 μl of Bst DNA Polymerase Large-Fragment stock enzyme (New England Biolabs, Beverly, Mass. (NEB catalog #M0275L, 8,000 Units/ml)) and 1.0 μl of 10× Thermopol buffer (New England Biolabs, Beverly, Mass.) to a final volume of 10 μl.

Solution A was added into a well of a 96-well thermocycler plate, such that there were 4 wells comprising a solution A in the 96 well plate, each well comprising a different primer set, a different template, and/or a different template concentration. The plate wells were sealed, and the plate was placed into an ABI 9700 thermocycler (Applied Biosystems, Foster City, Calif.). The plate was heated at 94° C. for 3 minutes. Following the heating step, the plate was cooled to 4° C. for 1 to 5 minutes. The plate was then heated to 50° C. and the wells were unsealed. Once the wells were unsealed, 10 μl of solution B was added with gentle mixing. The wells were resealed, and the plate was maintained at 50° C. for 5 hours.

The reaction composition in each well comprised the following:

1× ThermoPol (20 mM Tris-HCl pH 8.8, 10 mM KCl, 10 mM (NH₄)SO₄, 2 mM MgSO₄, 0.1% Triton X-100)

4% neat DMSO

50 μM Primer

1 mM dNTPs (0.25 mM each: dATP, dCTP, dGTP and dTTP)

0.35 U/μl Bst DNA Polymerase (Large-Fragment)

B. ceres lysate (0 μl, 0.01 μl, 0.1 μl, or 1.0 μl)

Following amplification, the amplification products of each amplification reaction were diluted 25 fold in water, and a 2.5 μl volume of each diluted amplification product was added to a separate 10 μl TaqMan assay. The TaqMan assay was otherwise carried out as described in Example 4. A bacterial 16S target was used as the TaqMan probe: The results of the TaqMan assays are shown below.

| Volume of lysate | | | | |
|---|---|---|---|---|
| (μl) | No amp C_{T} | amp C_{T} | ΔC_{T} | Fold Difference |
| 0 | 33.16 (0.85) | 27.35 (0.01) | 5.82 | 56 |
| 0.01 | 26.80 (0.06) | 19.32 (0.04) | 7.48 | 178 |
| 0.1 | 25.91 (0.06) | 16.83 (0.05) | 9.08 | 541 |
| 1.0 | 22.58 (0.04) | 15.57 (0.04) | 7.01 | 129 |

( ) = standard deviation of replicate C_{T} values

### EXAMPLE 17

Amplification of Nucleic Acids from Buccal Swab Lysates (Human)

Amplification reactions were used to amplify genomic DNA from buccal swab lysates. A buccal swab was placed in 500 μl of 0.1 M NaOH and incubated for 5 minutes at room temperature. Following incubation, 50 μl of TE was added and the tube was vortexed for 3 seconds. Following vortexing, aliquots were removed for amplification. For a no template control, 50 μl of TE was stored on ice.

Four different reaction compositions were prepared as follows:

Four separate solutions of solution A were prepared by adding 5 μl of a 500 μM stock of primer set DR10D (SSN₈), 2 μl of neat DMSO (American Type Culture Collection (ATCC), Rockville, Md.), 0.5 μl of 100 mM dNTPs (25 mM each: dATP, dCTP, dGTP and dTTP) (Applied Biosystems, Foster City, Calif.), 4 μl of 10× Thermopol buffer (New England Biolabs, Beverly, Mass.), and an amount of buccal swab lysate as discussed below, to a final solution volume of 40 μl. The first solution A comprised the no template control; the second solution A comprised 1.0 μl of buccal swab lysate; the third solution A comprised 2.0 μl of buccal swab lysate; and the fourth solution A comprised 5.0 μl of buccal swab lysate.

Solution B was prepared by adding 2.2 μl of Bst DNA Polymerase Large-Fragment stock enzyme (New England Biolabs, Beverly, Mass. (NEB catalog #M0275L, 8,000 Units/ml)) and 1.0 μl of 10× Thermopol buffer (New England Biolabs, Beverly, Mass.) to a final volume of 10 μl.

Solution A was added into a well of a 96-well thermocycler plate, such that there were 28 wells comprising a solution A in the 96 well plate, each well comprising a different reaction composition. The plate wells were sealed, and the plate was placed into an ABI 9700 thermocycler (Applied Biosystems, Foster City, Calif.). The plate was heated at 94° C. for 3 minutes. Following the heating step, the plate was cooled to 4° C. for 1 to 5 minutes. The plate was then heated to 50° C. and the wells were unsealed. Once the wells were unsealed, 10 μl of solution B was added with gentle mixing. The wells were resealed, and the plate was maintained at 50° C. for 5 hours.

The reaction composition in each well comprised the following:

1× ThermoPol (20 mM Tris-HCl pH 8.8, 10 mM KCl, 10 mM (NH₄)SO₄, 2 mM MgSO₄, 0.1% Triton X-100)

4% neat DMSO

50 μM Primer

1 mM dNTPs (0.25 mM each: dATP, dCTP, dGTP and dTTP)

0.35 U/μl Bst DNA Polymerase (Large-Fragment) buccal swab lysate (0 μl, 1.0 μl, 2.0 μl, or 5.0 μl)

Following amplification, the amplification products were diluted 25 fold in water and a 2.5 μl volume of the diluted amplification products was added to a 10 μl TaqMan assay. Two separate TaqMan assays were performed for each set of reaction products. The TaqMan assays were otherwise carried out as described in Example 4. The TaqMan probes used in the TaqMan assays were either Chromosome 6.2 or Chromosome 6.11. The Chromosome 6.11 probe and the Chromosome 6.2 probe are described in Example 10 and FIG. 10. GC content for the genomic region surrounding the position that hybridizes to the TaqMan probe of a particular assay was calculated. Results of the TaqMan assay are shown below:

| Volume of lysate | | | | |
|---|---|---|---|---|
| (μl) | No amp C_{T} | amp C_{T} | ΔC_{T} | Fold Difference |
| Chromosome 6.2 | | | | |
| (50% GC) | | | | |
| 1.0 | 36.65 (0.14) | 28.77 (0.12) | 7.88 | 235 |
| 2.0 | 35.68 (0.29) | 28.68 (0.03) | 7.00 | 128 |
| 5.0 | 33.60 (0.60) | 28.34 (0.05) | 5.26 | 38 |
| Chromosome 6.11 | | | | |
| (35% GC) | | | | |
| 1.0 | 37.81 (1.62) | 30.94 (0.19) | 6.87 | 117 |
| 2.0 | 37.64 (1.23) | 30.19 (0.30) | 7.45 | 175 |
| 5.0 | 33.98 (0.08) | 29.22 (0.04) | 4.76 | 27 |

### EXAMPLE 18

Amplification of Nucleic Acids from Crude Blood Lysate

5 μl of fresh human blood from a healthy adult male was taken for analysis. 25 μl of Phosphate Buffered Saline (PBS) was added to the blood. After adding the PBS, 35 μl of KOH was added and mixed by pipeting. Following addition of KOH, 35 μl of Tris was added and the final volume of the blood preparation was brought to 100 μl.

Three different reaction compositions were prepared as follows:

Three separate solutions of solution A were prepared by adding 5 μl of a 500 μM stock of primer set DR10D (SSN₈), 2 μl of neat DMSO (American Type Culture Collection (ATCC), Rockville, Md.), 0.5 μl of 100 mM dNTPs (25 mM each: dATP, dCTP, dGTP and dTTP) (Applied Biosystems, Foster City, Calif.), and 4 μl of 10× Thermopol buffer (New England Biolabs, Beverly, Mass.), and an amount of blood preparation as discussed below, to a final solution volume of 40 μl. The first solution A contained 0.05 μl of the blood preparation. The second solution A contained 0.125 μl of the blood preparation. The third solution A contained 0.5 μl of the blood preparation.

Solution B was prepared by adding 2.2 μl of Bst DNA Polymerase Large-Fragment stock enzyme (New England Biolabs, Beverly, Mass. (NEB catalog #M0275L, 8,000 Units/ml)) and 1.0 μl of 10× Thermopol buffer (New England Biolabs, Beverly, Mass.) to a final volume of 10 μl.

Solution A was added into a well of a 96-well thermocycler plate, such that there were 3 wells comprising a solution A in the 96 well plate, each well comprising a different reaction composition. The plate wells were sealed, and the plate was placed into an ABI 9700 thermocycler (Applied Biosystems, Foster City, Calif.). The plate was heated at 94° C. for 3 minutes. Following the heating step, the plate was cooled to 4° C. for 1 to 5 minutes. The plate was then heated to 50° C. and the wells were unsealed. Once the wells were unsealed, 10 μl of solution B was added with gentle mixing. The wells were resealed, and the plate was maintained at 50° C. for 5 hours.

The reaction composition in each well comprised the following:

1× ThermoPol (20 mM Tris-HCl pH 8.8, 10 mM KCl, 10 mM (NH₄)SO₄, 2 mM MgSO₄, 0.1% Triton X-100)

4% neat DMSO

50 μM Primer

1 mM dNTPs (0.25 mM each: dATP, dCTP, dGTP and dTTP)

0.35 U/μl Bst DNA Polymerase (Large-Fragment)

blood preparation (0.05 μl, 0.125 μl, or 0.5 μl)

Following amplification, the amplification products of each reaction were evaluated using TaqMan assays as described in Example 4. The probes used for the TaqMan assays were an RNase P probe, a Chromosome 6.9 probe, and a Chromosome 6.14 probe. The RNase P probe is described in Example 4. The Chromosome 6.9 probe is described in Example 10 and FIG. 10. The Chromosome 6.14 probe is also described in Example 10 and FIG. 10. GC content for the genomic region surrounding the position that hybridizes to the TaqMan probe of a particular assay was also calculated. Results of the TaqMan assays are shown below:

Summary of Amplification Results Using Blood as a Template

| | RNase P | | 6.9 | 6.14 |
|---|---|---|---|---|
| Percent GC content\hat{ } | 61% | | 70% | 35% |
| 0.5 μl blood | 107 | ng/μl* | 172 ng/μl | 12 ng/μl |
| 0.125 μl blood | 97 | ng/μl | 123 ng/μl | 6 ng/μl |
| 0.05 μl blood | 0 | ng/μl | 0 ng/μl | 0 ng/μl |

*Results are quantitated as ng of DNA per μl of amplification reaction.
\hat{ }Percent GC content refers to the percent GC content at each TaqMan target sequence.

### EXAMPLE 19

Effect of Betaine on Amplification Using Exemplary Primer Sets

Six different primer sets were used to test the effect of betaine on amplification reactions. The primer sets used were DR3, DR3B, DR10D, DR11D, DR33E, and DR77E. Different temperatures and betaine concentrations were tested in the reaction compositions.

Separate solutions of solution A were prepared by adding 5 μl of a 500 μM stock of one of primers DR3, DR3B, DR10D, DR11D, DR33E, and DR77E, 2 μl of neat DMSO (American Type Culture Collection (ATCC), Rockville, Md.), 0.5 μl of 100 mM dNTPs (25 mM each: dATP, dCTP, dGTP and dTTP) (Applied Biosystems, Foster City, Calif.), 4 μl of 10× Thermopol buffer (New England Biolabs, Beverly, Mass.), 1 μl of a 10 ng/μl stock of Human genomic DNA template (Clontech, Palo Alto, Calif.), and an amount of Betaine as discussed below, to a final solution volume of 40 μl. For each primer set, one solution A was formed comprising 0 μl of a 5M stock of Betaine (Sigma. Aldrich, St. Louis, Mo.); one solution A was formed comprising 5 μl of a 5M stock of Betaine (Sigma Aldrich, St. Louis, Mo.); one solution A was formed comprising 10 μl of a 5M stock of Betaine (Sigma Aldrich, St. Louis, Mo.); one solution A was formed comprising 12 μl of a 5M stock of Betaine (Sigma Aldrich, St. Louis, Mo.); one solution A was formed comprising 15 μl of a 5M stock of Betaine (Sigma Aldrich, St. Louis, Mo.); one solution A was formed comprising 18 μl of a 5M stock of Betaine (Sigma Aldrich, St. Louis, Mo.); and one solution A was formed comprising 20 μl of a 5M stock of Betaine (Sigma Aldrich, St. Louis, Mo.).

Each Solution B was prepared by adding 4.7 μl of Bst DNA Polymerase Large-Fragment stock enzyme (New England Biolabs, Beverly, Mass. (NEB catalog #M0275L, 8,000 Units/ml)), 1.0 μl of 10× Thermopol buffer (New England Biolabs, Beverly, Mass.) to a final volume of 10 μl.

For each amplification reaction, Solution A was added into a well of a 96-well thermocycler plate, such that there were 36 wells comprising a solution A in the 96 well plate, each well comprising a different reaction composition. The plate wells were sealed, and the plate was placed into an ABI 9700 thermocycler (Applied Biosystems, Foster City, Calif.). The plate was heated at 94° C. for 3 minutes. Following the heating step, the plate was cooled to 4° C. for 1 to 5 minutes. The plate was then heated to 50° C. and the wells were unsealed. Once the wells were unsealed, 10 μl of solution B was added with gentle mixing. The wells were resealed.

Each different reaction composition was prepared in duplicate. The first plate of each different reaction composition was maintained at 47° C. for at least 16 hours after the wells were resealed. The second plate of each different reaction composition was maintained at 50° C. for at least 16 hours after the wells were resealed.

The reaction composition in each well comprised the following:

1× ThermoPol (20 mM Tris-HCl pH 8.8 @25° C., 10 mM KCl, 10 mM (NH₄)SO₄, 2 mM MgSO₄, 0.1% Triton X-100)

4% DMSO

50 μM Primer

1 mM dNTPs (0.25 mM each: dATP, dCTP, dGTP and dTTP)

0.75 U/μl Bst DNA Polymerase (Large-Fragment)

10 ng of Clontech Human genomic DNA (Clontech, Palo Alto, Calif.)

0 M, 0.5 M, 1 M, 1.2 M, 1.5 M, 1.8 M, or 2 M Betaine

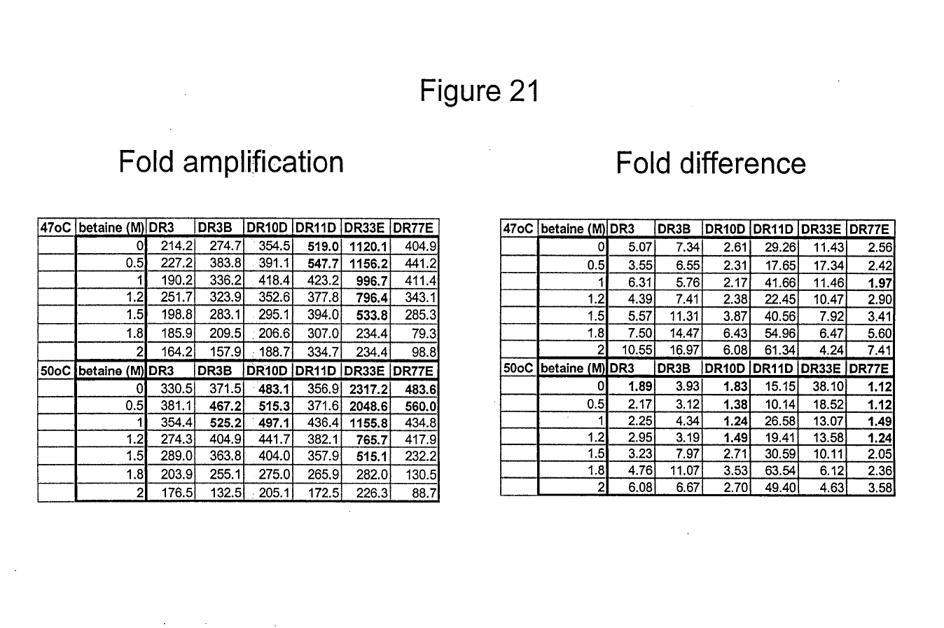

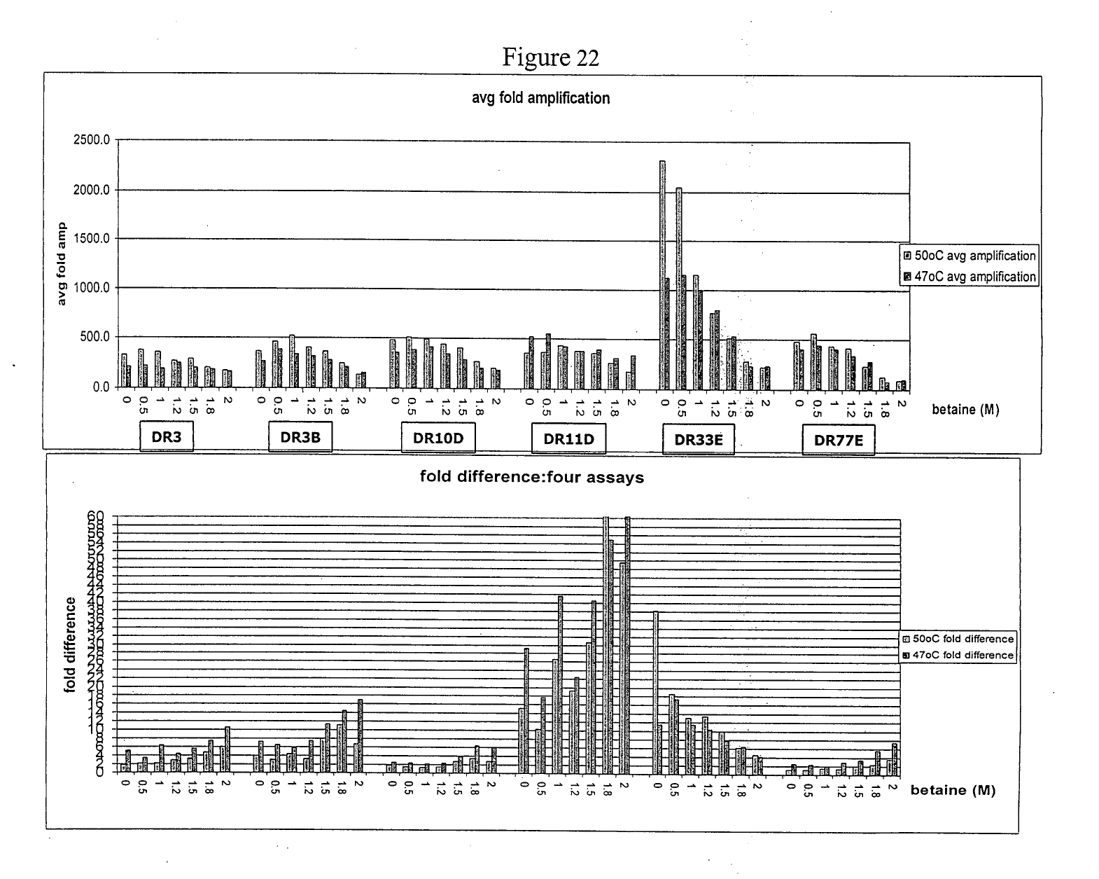

The amplification products of each amplification reaction were evaluated using TaqMan assays as described in Example 4. Specifically, reaction products from each amplification reaction were evaluated using TaqMan assays specific for four positions. The four TaqMan probes that were used were the RNase P probe, the Chromosome 6.2 probe, the Chromosome 6.9 probe, and the Chromosome 6.14 probe. The RNase P probe is described in Example 4. The Chromosome 6.2 probe, the Chromosome 6.9 probe, and the Chromosome 6.14 probe are described in Example 10 and FIG. 10. For each reaction, a fold difference and average fold amplification were calculated based on individual measurements for the four TaqMan assays, as is shown in FIGS. 21 and 22.

1. A method of amplifying a plurality of target nucleic acid sequences comprising:
forming a reaction composition comprising (a) a plurality of target nucleic acid sequences, (b) at least one set of primers, and (c) at least one polymerase;
incubating the reaction composition under conditions wherein one or more of the plurality of target nucleic acid sequences are amplified;
wherein at least one of the at least one set of primers comprises a plurality of primers, wherein each primer of the plurality of primers comprises at least one designed portion and at least one random portion;
wherein one of the at least one designed portions consists of two nucleotides at the 5′ portion of the primer.

2. The method of claim 1, wherein at least one of the at least one random portions comprises eight random nucleotides.

3. The method of claim 1, wherein the at least one polymerase is selected from at least one of phi29 DNA polymerase, taq polymerase, stoffel fragment, and Bst DNA polymerase.

4. The method of claim 1, wherein the two nucleotides at the 5′ portion of the primer comprise pyrimidines.

5. The method of claim 4, wherein at least one of the at least one set of primers comprises primers of the sequence CTN₈.

6. The method of claim 1, wherein the two nucleotides at the 5′ portion of the primer comprise purines.

7. The method of claim 6, wherein at least one of the at least one set of primers comprises primers of the sequence GAN₈.

8. The method of claim 1 wherein at least one of the at least one set of primers comprises primers of the sequence SSN₈.

9. The method of claim 1, wherein the at least one DNA polymerase is inactive and is subsequently activated at a given temperature.

10. The method of claim 1, wherein at least one of the at least one random portions is between 6 nucleotides and 9 nucleotides in length.

11. The method of claim 1, wherein the plurality of target nucleic acid sequences comprises genomic DNA.

12. The method of claim 1, wherein the plurality of target nucleic acid sequences are amplified under isothermal reaction conditions.

13. The method of claim 12, wherein the isothermic conditions comprise a temperature of about 50° C.

14. The method of claim 1, wherein the plurality of target nucleic acid sequences comprises mitochondrial DNA.

15. The method of claim 1, further comprising treating at least one of the plurality of target nucleic acid sequences with a modifying agent before forming the reaction composition.

16. The method of claim 15, wherein the modifying agent is bisulfite.

17. The method of claim 1, wherein the plurality of target nucleic acid sequences comprises one or more forensic markers.

18. The method of claim 1, wherein the reaction composition has only one set of primers.

19. A composition for amplifying a plurality of target nucleic acid sequences comprising:
(a) a plurality of target nucleic acid sequences;
(b) at least one set of primers; and
(c) at least one DNA polymerase;
wherein at least one of the at least one set of primers comprises a plurality of primers, wherein each primer of the plurality of primers comprises at least one designed portion and at least one random portion;
wherein one of the at least one designed portions consists of two nucleotides at the 5′ portion of the primer.

20. The composition of claim 19, wherein one of the at least one random portions comprises eight random nucleotides.

21. The composition of claim 19, wherein the at least one DNA polymerase is selected from at least one of phi29 DNA polymerase, taq polymerase, stoffel fragment, and Bst DNA polymerase.

22. The composition of claim 19, wherein the two nucleotides at the 5′ portion of the primer comprise pyrimidines.

23. The composition of claim 22, wherein at least one of the at least one sets of primers comprises primers of the sequence CTN₈.

24. The composition of claim 19, wherein the two nucleotides at the 5′ portion of the primer comprise purines.

25. The composition of claim 24, wherein one of the at least one primer sets comprises primers of the sequence GAN₈.

26. The method of claim 19 wherein at least one of the at least one set of primers comprises primers of the sequence SSN₈.

27. The composition of claim 19, wherein one of the at least one random portions is between 6 nucleotides and 9 nucleotides in length.

28. The composition of claim 19, wherein the plurality of target nucleic acid sequences comprises genomic DNA.

29. The composition of claim 19, wherein the plurality of target nucleic acid sequences comprises mitochondrial DNA.

30. The composition of claim 19, wherein the plurality of target nucleic acid sequences comprises one or more forensic markers.

31. The composition of claim 19, wherein the reaction composition comprises one set of primers.

32. A method of amplifying a plurality of target nucleic acid sequences comprising:
forming a reaction composition comprising (a) a plurality of target nucleic acid sequences comprising a first position and a second position, (b) at least one set of primers, and (c) at least one DNA polymerase;
incubating the reaction composition under amplification conditions wherein one or more of the plurality of target nucleic acid sequences are amplified to form amplification products;
wherein at least one of the at least one set of primers comprises a plurality of primers, wherein each primer of the plurality of primers comprises at least one designed portion and at least one random portion;
wherein the at least one set of primers produces a first amplification product comprising the first position and a second amplification product comprising the second position;
wherein the amount of first amplification product produced with the at least one set of primers is greater than the amount of first amplification product produced with random primers incubated under the amplification conditions with the plurality of target nucleic acid sequences and the at least one polymerase; and
wherein the amount of second amplification product produced with the at least one set of primers is greater than the amount of second amplification product produced with random primers incubated under the amplification conditions with the plurality of target nucleic acid sequences and the at least one polymerase.

33. The method of claim 32, wherein the first position comprises a telomere region.

34. The method of claim 32, wherein the first position comprises a centromere region.

35. The method of claim 32, wherein the first set of primers comprises primers of the sequence N₃T₄N₃ (SEQ ID NO: 5).

36. A method of determining similarity between a plurality of target nucleic acid sequences from one or more sources and one or more reference sequences comprising:
forming a reaction composition comprising (a) a plurality of target nucleic acid sequences from one or more sources, (b) at least one set of primers, and (c) at least one DNA polymerase;
incubating the reaction composition under conditions wherein one or more of the plurality of target nucleic acid sequences are amplified to form amplification products;
wherein at least one of the at least one set of primers comprises a plurality of primers, wherein each primer of the plurality of primers comprises at least one designed portion and at least one random portion;
wherein one of the at least one designed portions consists of two nucleotides at the 5′ portion of the primer;
comparing sequence information of the amplification products with sequence information of the one or more reference sequences to identify similarities and differences between the sequence information of the amplification products and the sequence information of the one or more reference sequences; wherein each of the one or more reference sequences comprises a plurality of nucleic acid sequences;
determining the similarity between a plurality of target nucleic acid sequences from one or more sources and the one or more reference sequences based on the identified similarities and differences.

37. The method of claim 36, wherein the comparing sequence information of the amplification products with the sequence information of the one or more reference sequences comprises comparing the sequence of the amplification products at a given nucleic acid polymorphic site to the sequence of the one or more reference sequences at the given nucleic acid polymorphic site.

38. The method of claim 37, wherein the nucleic acid polymorphic site comprises a single nucleotide polymorphism.

39. The method of claim 36, further comprising determining whether one or more of the plurality of target nucleic acid sequences is the same as one of the one or more reference sequences.

40. A method of amplifying a plurality of target nucleic acid sequences comprising:
forming a reaction composition comprising (a) a plurality of target nucleic acid sequences, (b) at least one set of primers, and (c) at least one DNA polymerase;
incubating the reaction composition under conditions wherein one or more of the plurality of target nucleic acid sequences are amplified;
wherein at least one of the at least one set of primers comprises a plurality of primers, wherein each primer of the plurality of primers comprises at least one designed portion and at least one random portion;
wherein one of the at least one designed portions comprises the 5′ portion of the primer.

41. A method of amplifying a plurality of target nucleic acid sequences comprising:
forming a reaction composition comprising (a) a plurality of target nucleic acid sequences, (b) at least one set of primers, and (c) at least one DNA polymerase;
incubating the reaction composition under conditions wherein one or more of the plurality of target nucleic acid sequences are amplified;
wherein at least one of the at least one set of primers comprises a plurality of primers, wherein each primer of the plurality of primers comprises at least one designed portion and at least one random portion;
wherein one of the at least one designed portions comprises the 3′ portion of the primer.

42. A method of amplifying a plurality of target nucleic acid sequences comprising:
forming a reaction composition comprising (a) a plurality of target nucleic acid sequences, (b) at least one set of primers, and (c) at least one DNA polymerase;
incubating the reaction composition under conditions wherein one or more of the plurality of target nucleic acid sequences are amplified;
wherein at least one of the at least one set of primers comprises a plurality of primers, wherein each primer of the plurality of primers comprises at least one designed portion and at least one random portion;
wherein at least one of the at least one designed portions is not a constant portion.
